SEQUENCE LISTING (2 sequences)

SEQ ID NO: 1 - protein, 119 aa, synthetic construct [sequence omitted]

SEQ ID NO: 2 - protein, 107 aa, synthetic construct [sequence omitted]

(19) United States
(12) Patent Application Publication
(10) Pub. No.: US 2025/0154243 A1
(43) Pub. Date: May 15, 2025

(54) COMBINATION TREATMENT OF CHRONIC MYELOMONOCYTIC LEUKEMIA IN PATIENTS WITH RAS PATHWAY MUTATIONS

(71) Applicants: TARAN THERAPEUTICS, INC., Toms River, NJ (US); THE UNIVERSITY OF ADELAIDE, Adelaide (AU)

(72) Inventors: Cameron DURRANT, Bella Collina, FL (US); Daniel THOMAS, Adelaide (AU)

(73) Assignees: TARAN THERAPEUTICS, INC., Toms River, NJ (US); THE UNIVERSITY OF ADELAIDE, Adelaide, SA (AU)

(21) Appl. No.: 18/889,359

(22) Filed: Sep. 18, 2024

Related U.S. Application Data
(63) Continuation-in-part of application No. 18/604,466, filed on Mar. 13, 2024.
Application No. 18/604,466 is a continuation of application No. PCT/US24/19755, filed on Mar. 13, 2024.
(60) Provisional application No. 63/595,285, filed on Nov. 1, 2023.
(60) Provisional application No. 63/452,147, filed on Mar. 14, 2023.
(60) Provisional application No. 63/595,285, filed on Nov. 1, 2023.
(60) Provisional application No. 63/452,147, filed on Mar. 14, 2023.
(60) Provisional application No. 63/595,285, filed on Nov. 1, 2023.

(51) Int. Cl. C07K 16/24; A61K 31/17; A61K 31/706; A61K 31/7068; A61K 39/00; A61K 45/06; A61P 35/02; C12Q 1/6886

(52) U.S. Cl. CPC C07K 16/243; A61K 31/17; A61K 31/706; A61K 31/7068; A61K 45/06; A61P 35/02; C12Q 1/6886; A61K 2039/505; A61K 2039/54; A61K 2039/545; C12Q 2600/106

(57) ABSTRACT

Provided herein are methods for treating a subject having chronic myelomonocytic leukemia (CMML), the method comprising: (a) identifying a RAS pathway mutation in tumor cells of the subject, wherein the RAS pathway mutation is a NRAS, KRAS, PTPN-11 and/or CBL mutation; (b) identifying a dominant CBL mutation of CBL variant allele frequency of from <5% to >10%; and (c) administering to the subject identified in step (a) a therapeutically effective amount of an anti-hGM-CSF antibody. Also provided herein are methods for treating a subject having chronic myelomonocytic leukemia (CMML), the method comprising: (a) identifying a RAS pathway mutation in tumor cells of the subject, wherein the RAS pathway mutation is a NRAS, KRAS, PTPN-11 and/or CBL mutation; (b) identifying a dominant CBL mutation of CBL variant allele frequency of from <5% to >10%; and (c) administering to the subject identified in step (a) a therapeutically effective amount of an anti-hGM-CSF antibody lenzilumab and a therapeutically effective amount of a second therapeutic agent. The subject may have a RAS pathway mutation or a RAS pathway mutation and at least one TET2 mutation identified in the tumor cells, an increased percentage of CD116 and CD131 in CD34⁺ stem and progenitor cells in the subject compared to a healthy subject and/or an increased percentage of CD14⁺ cells in the subject compared to a healthy subject. A therapeutically effective amount of a hypomethylating agent or hydroxyurea may be further administered according to the provided methods.

### CROSS-REFERENCE TO RELATED APPLICATIONS

This application claims priority to and the benefit of U.S. Provisional Application No. 63/595,285, filed on Nov. 1, 2023, and is a continuation-in-part application of U.S. application Ser. No. 18/604,466, filed Mar. 13, 2024, which claims priority to and the benefit of U.S. Provisional Application No. 63/452,147, filed on Mar. 14, 2023 and U.S. Provisional Application No. 63/595,285, filed on Nov. 1, 2023, and of PCT International Application No. PCT/US24/19755, filed Mar. 13, 2024, which claims priority to and the benefit of and U.S. Provisional Application No. 63/452,147, filed on Mar. 14, 2023 and U.S. Provisional Application No. 63/595,285, filed on Nov. 1, 2023, each of each of which is hereby incorporated by reference in their entireties.

### INCORPORATION-BY-REFERENCE OF SEQUENCE LISTING

The Sequence Listing XML associated with this application is provided electronically in XML file format and is hereby incorporated by reference into the specification. The name of the XML file containing the Sequence Listing XML is “P-623428-US1_ST26.xml”. The XML file is 3,945 bytes, created on Dec. 17, 2024, and is being submitted electronically via USPTO Patent Center.

### FIELD OF THE INVENTION

The invention relates to methods for treating a subject having chronic myelomonocytic leukemia (CMML), the method comprising: (a) identifying a RAS pathway mutation in tumor cells of the subject, wherein the RAS pathway mutation is a NRAS, KRAS, PTPN-11 and/or CBL mutation; (b) identifying a dominant CBL mutation of CBL variant allele frequency of from <5% to >10%; and (c) administering to the subject identified in in steps (a) and (b) or solely in step (b) a therapeutically effective amount of an anti-hGM-CSF antibody. In some aspects, the methods for treating the subject having CMML further comprise administering to the subject a therapeutically effective amount of an anti-hGM-CSF antibody and a therapeutically effective amount of a second therapeutic agent, wherein the second therapeutic agent is a hypomethylating agent or a chemotherapy drug. The invention further relates to methods for treating a subject having CMML, the methods comprising: (a) identifying a RAS pathway mutation in tumor cells of the subject, wherein the RAS pathway mutation is a NRAS, KRAS, PTPN-11 and/or CBL mutation; (b) identifying a dominant CBL mutation of CBL variant allele frequency of from <5% to >10%; and (c) administering to the subject identified in steps (a) and (b) or solely in step (b) a therapeutically effective amount of an anti-hGM-CSF antibody and a therapeutically effective amount of a second therapeutic agent. In some aspects, the a second therapeutic agent is a hypomethylating agent or a chemotherapy drug.

### BACKGROUND OF THE INVENTION

Chronic myelomonocytic leukemia (CMML) is a rare, aggressive cancer for which no targeted therapy exists. Standard of care (SOC) includes hypomethylating agents such as azacitidine (A) and decitabine (D), with complete and partial response (CR and PR) rates ranging between 7-21%. The pro-inflammatory cytokine granulocyte-macrophage colony-stimulating factor (GM-CSF) can play a role in stimulating leukemic cell proliferation.

Accordingly, there remains a critical need for developing improved compositions and methods for treatment of CMML, for achieving complete response (CR) plus partial response (PR) that is higher than the CR and PR rate demonstrated with sole administration of a hypomethylating agent, and for improving survival and progression-free survival after treatment compared to survival and progression-free survival after administration of a hypomethylating agent alone.

### SUMMARY OF THE INVENTION

In one aspect, provided herein is a method for treating a subject having chronic myelomonocytic leukemia (CMML), the method comprising: (a) identifying a RAS pathway mutation in tumor cells of the subject, wherein the RAS pathway mutation is a NRAS, KRAS, PTPN-11 and/or CBL mutation; (b) identifying a dominant CBL mutation of CBL variant allele frequency of from <5% to >10%; and (c) administering to the subject identified in steps (a) and (b) or solely in step (b) a therapeutically effective amount of an anti-hGM-CSF antibody.

In another aspect, provided herein is a method for treating a subject having chronic myelomonocytic leukemia (CMML), the method comprising: (a) identifying a RAS pathway mutation in tumor cells of the subject, wherein the RAS pathway mutation is a NRAS, KRAS, PTPN-11 and/or CBL mutation; (b) identifying a dominant CBL mutation of CBL variant allele frequency of from <5% to >10%; and (c) administering to the subject identified in steps (a) and (b) or solely in step (b) a therapeutically effective amount of an anti-hGM-CSF antibody and a therapeutically effective amount of a second therapeutic agent.

In one aspect, provided herein is a method for treating a subject having chronic myelomonocytic leukemia (CMML), the method comprising: (a) identifying a RAS pathway mutation in tumor cells of the subject, wherein the RAS pathway mutation is a NRAS, KRAS, PTPN-11 and/or CBL mutation; and (b) administering to the subject identified in step (a) a therapeutically effective amount of an anti-hGM-CSF antibody.

In another aspect, provided herein is a method for treating a subject having chronic myelomonocytic leukemia (CMML), the method comprising: (a) identifying a RAS pathway mutation in tumor cells of the subject, wherein the RAS pathway mutation is a NRAS, KRAS, PTPN-11 and/or CBL mutation; and (b) administering to the subject identified in step (a) a therapeutically effective amount of an anti-hGM-CSF antibody and a therapeutically effective amount of a hypomethylating agent or a chemotherapy drug.

Other features and advantages of the present invention will become apparent from the following detailed description examples and figures. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure, the inventions of which can be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIGS. 1A-1B show the results of Phase II DNA methyltransferase inhibitor trials (FIG. 1A) and the morphologie criteria for diagnosis of CMML including identification and quantitation of blast cells and blast-equivalent cells, including promonocytes (FIG. 1B).

FIGS. 2A-2C show the baseline characteristics of the lenzilumab cohort in the studies of Example 1: age (FIG. 2A), sex at birth (FIG. 2B), and spleen size in cm (FIG. 2C).

FIGS. 3A-3B show the baseline characteristics of the lenzilumab cohort in the studies of Example 1: cytogenetics (FIG. 3A) and bone marrow (BM) Blast % (FIG. 3B).

FIGS. 4A-4B show the baseline characteristics of the lenzilumab cohort in the studies of Example 1: whether a subject is transfusion dependent (FIG. 4A) and the amount of hemoglobin (g/L) in a subject's blood (FIG. 4B).

FIGS. 5A-5B show the baseline characteristics of the lenzilumab cohort in the studies of Example 1: white blood cell count (FIG. 5A) and monocytes (FIG. 5B).

FIG. 6 shows in tabular format the baseline characteristics of the lenzilumab cohort in the studies of Example 1: the gene mutation profile, including NRAS, KRAS, PTPN-11 and/or CBL mutations and TET2 mutations, of 11 patients.

FIG. 7 shows in a pie chart format the genes mutated in the lenzilumab cohort. Cbl mutations are frequent, as are TET2 mutations.

FIGS. 8A-8B show the baseline characteristics of the lenzilumab cohort in the studies of Example 1: CPSS-MOL Score (FIG. 8A) and the constitutional symptoms of fatigue, weight loss and night sweats (FIG. 8B). CPSS-MOL Score is a CMML-specific prognostic scoring system (CPSS) that incorporates molecular genetic data resulting in a 4-level integrated clinical/pathological/genetic risk stratification tool.

FIG. 9 shows excellent response rates in lenzilumab-azacitidine (Lenz-Aza) combination therapy: 83% CR rate, 100% ORR (CR+Clinical Benefit). Clinical benefit includes platelet response.

FIG. 10 shows in a tabular format the baseline characteristics of the lenzilumab cohort in the studies of Example 1: CPSS-MOL Score; blast %, Hb/WCC/Platelet (Plt)×10⁹/L and monocyte count; the response after 3 cycles of combination therapy (lenz+aza) of blast %, Hb/WCC/Platelet (Plt)×10⁹/L and monocyte count; the best response (complete remission or platelet response; and the Myeloproliferative Neoplasm (MPN) total symptom score.

FIG. 11 shows the Savona criteria for myelodysplastic/myeloproliferative neoplasms (MPN/MDS) overlap syndromes in tabular format. Savona et al. (2015) proposed response assessment guidelines to harmonize future clinical trials with the principal objective of establishing suitable treatment algorithms based on the recommendations of an international panel comprising laboratory and clinical experts in MDS/MPN involving three independent academic MDS/MPN workshops (two in 2013 and one in 2014).

FIG. 12 shows the superior early response data of Lenz+Aza combination therapy for CMML compared to published literature for CMML treatment with DNA methyltransferase inhibitors (DNMTi) alone.

FIG. 13 shows high CR response rate (%) obtained from Lenz+Aza combination therapy in high risk CMML patients.

FIG. 14 shows the robust improvement in quality of life based on the MPN symptoms assessment form: total symptom score compared to baseline scores. The best responding symptoms were fatigue, weight loss, poor concentration, and inactivity.

FIG. 15 shows in tabular format certain serious adverse events that occurred in the lenzilumab cohort in the Lenz+Aza combination therapy for CMML.

FIG. 16 shows in tabular format the grade 3 & 4 adverse events of the lenzilumab cohort in the Lenz+Aza combination therapy for CMML.

FIG. 17 shows in tabular format the patient characteristics from a prospective phase II trial of azacytidine therapy in CMML patients (Drummond et al, Leukemia 2014). 32 patients with CMML-1 (70%) or CMML-2 (27%) treated with azacitidine 75 mg/m² were of a median age of 70; had genetic mutations (TET2 63%, ASXL1 38%, EZH2 4%, CBL 8%, NRAS 5%); and median WCC 15.9×10⁹/L.

FIGS. 18A-18B show lenzilumab in addition to Azacitidine improves complete response rates in chronic myelomonocytic leukemia. FIG. 18A shows a Swimmer plot showing ongoing treatment of patients with RAS-pathway mutations on LENZ/AZA arm of the study. Black arrow indicates patient has not withdrawn/progressed. FIG. 18B shows column graphs showing decreased bone marrow blast % with LENZ+AZA treatment assessed after 3, 6 and 2 months of combination treatment. P values reflect unpaired students t-test for groups as shown.

FIGS. 19A-19B show decreased size of RAS-pathway mutant clones after lenzilumab+Azacitidine. FIG. 19A shows a Circos plot showing all variant somatic mutations identified at screening in CMML patients. FIG. 19B shows column graphs showing decrease of KRAS and CBL VAF (%) in 4 individuals after LENZ+AZA treatment.

FIGS. 20A-20B show improvement in inflammatory parameters in CMML patients treated with lenzilumab and Azacitidine. FIG. 20A shows column graphs showing decrease in C-reactive protein and bone marrow GM-CSF after 3, 6 and 12 month cycles. FIG. 20B shows a heatmap showing hierarchical clustering of cytokine levels measured from CMML patient bone marrow interstitial fluid obtained at screening (screen) compared to age-matched healthy controls (HC). Two discrete clusters of CMML are apparent, INNATE-1 and INNATE-2. Patients with early response shown in orange blocks (sensitive).

FIGS. 21A-21B show a baseline gene mutation frequency in PREACHM (FIG. 21A) and the percentage of subjects showing VAF reduction >10% (FIG. 21B).

FIGS. 22A-2J show Fish plots showing decrease in VAF (in red) compared to parent clone (in blue). Line graphs showing decrease of RAS-pathway genes VAF % in 9 subjects after LENZ/AZA treatment in second row. Line graph tracking the percentage of blasts and WBC over time in third row.

FIG. 23 shows the treatment the CMML subjects received. Confirmed NRAS/KRAS or CBL Mutation at VAF≥3% (n=15) Lenzilumab (IV; 552 mg; day 1 & day 15 of cycle 1 and day 1 only for all subsequent cycles) and Azacitidine (SC; 75 mg/m² for 7 days). Confirmed TET2 mutation and without RAS-pathway mutation (n=4) Ascorbic acid (ASC) (IV; 30 g for 7 days (15 g for 1st dose only; 30 g thereafter if no evidence of tumor lysis syndrome); PO; 1.1 g on all other days) and Azacitidine (SC; 75 mg/m² for 7 days). If no mutation in either category, the screen was a fail.

FIGS. 24A-24B show Lenzilumab in addition to Azacitidine improves complete response rates in CMML. FIG. 24A shows a Swimmer plot showing ongoing treatment of patients with RAS-pathway mutations on LENZ/AZA arm of the study. Black arrow indicates patient has not progressed. FIG. 24B shows column graphs showing decreased bone marrow blast % with LENZ/AZA treatment assessed after 3, 6, 12 and 24 months of combination treatment. P values reflect unpaired students t-test for groups as shown.

FIGS. 25A-25C show durable responses with ongoing CR or marrow CR (IWG criteria) for patients in LENZ-AZA cohort (FIG. 25A) and durable decrease in white cell count (FIG. 25B) and monocyte count (FIG. 25C).

FIGS. 26A-26C show durable improvements in spleen length (FIG. 26A), platelet count (FIG. 26B), and hemoglobin (FIG. 25C). P values indicate unpaired Whitney-U non-parametric test.

FIG. 27 shows that patients with CBL-TET2 mutations are better responders to LENZ/AZA treatment. Within first 12 months of LENZ/AZA treatments, CMML patients with CBL-TET2 mutation showed excellent responses with a decrease in bone marrow blasts. CR-complete remission; mCR-marrow complete remission; PR-partial remission.

FIG. 28 shows a pro-inflammatory environment at baseline in CML patients: a Volcano plot illustrating cytokines levels in bone marrow plasma of CMML patients at baseline compared to age-matched healthy controls.

FIG. 29 shows that a cytokines profile at baseline can predict response to LENZ/AZA treatment: a Volcano plot representation of higher inflammatory cytokines levels in bone marrow plasma of patients resistant to LENZ/AZA treatment compared to patients responding to treatment.

FIGS. 30A-30B shows inflammatory parameters improvement in CMML patients after treatment with LENZ/AZA. FIG. 30A depicts column graphs showing a decrease in C-reactive protein from baseline and after 3, 6 and 12 months after LENZ/AZA treatment. FIG. 30B depicts column graphs showing bone marrow GM-CSF after 4, 7 and 13 cycles of LENZ/AZA treatment.

FIGS. 31A-31H show CBL mutants are associated with proliferative features, increased BM blast percentage, leukocytosis and splenomegaly. FIGS. 31A-31F show clinical characteristics of PREACH-M cohort at screening, stratified based on the detection of CBL mutation. FIG. 31G shows MD-, MP-CMML classification based on WCC^{α} FIG. 31H shows CMML-0, -1, -2 classifications based on BM blast percentage^{β}

### 2016 WHO Classification:

^{α}Based on WCC: MD-CMML WCC<13×10⁹/L, MP-CMML WCC>13×10⁹/L

^{β}Based on BM blast %: CMML-0 PB<2%, BM<5%; CMML-1 PB 2-4%, BM 5-9%, CMML-2 PB>5%, BM 10-19%.

Bars represent mean±standard error of mean. Mann-Whitney test used to determine statistical significance, where P<0.05 was statistically significant. *P<0.05.

[BM bone marrow; WCC white cell count; CRP C-reactive protein; MD-CMML myelodysplastic-CMML; MP-CMML myeloproliferative-CMML, wt wildtype, mut mutant]

FIGS. 32A-32E show CBL mutants frequently co-occur with TET2 mutants with strong correlation between CBL and TET2 VAF percentages and are associated with a complex subclonal architecture. FIG. 32A shows an oncoplot for the PREACH-M cohort (n=24). Mutation groups are shown in rows with each individual patient represented by a column. The presence of a mutation is indicated by the red or blue colored bars. Age category of the patients indicated by the black and grey bars and sex of patients by the green and gold bars. FIG. 32B shows co-occurrence of CBL mutant CMML (n=11) with other mutations by percentage, compared to CBL wildtype (n=13). FIG. 32D shows the number of cases where more than one variant of CBL, NRAS, KRAS or PTPN11 mutation was detected. FIG. 32C shows Pearson's correlation coefficient between TET2 and CBL VAF percentages, where P<0.05 was statistically significant. Pearson's correlation coefficient between the VAF percentages of CBL vs. RAS pathway genes. (data not shown) FIG. 32E shows VAF percentages of CBL variants detected in each patient with CBL mutation. [VAF variant allele frequency; V variant]

FIGS. 33A-33B show CMML have an increased percentage of CD116 and CD131 positive CD34⁺ stem and progenitor cells. FIG. 33A shows flow cytometry analysis of a representative CMML sample and healthy control stained with anti-CD45, -CD34, -CD14 and -CD16, and gating strategy used to define CD45⁺ mononuclear cells, CD34⁺ stem and progenitor cells, and CD14⁺ monocytes. FIG. 33B shows percentage of CD34⁺ stem and progenitor cells and CD14⁺ monocytes in CMML samples (n=4) vs. healthy control (n=2). The expression of CD114, CD115, CD116 and CD131 in CMML samples (n=4-6) vs. control (n=2-3) in CD45⁺, CD34⁺ and CD14⁺ subpopulations, expressed as percentage of positively stained cells (FIG. 33C) and MFI (FIG. 33D) compared to control (cord blood or peripheral blood mononuclear cells from healthy donors). Bars represent mean±standard deviation in FIG. 33B. Box and whiskers graphs were plotted with min and max in FIG. 33C and FIG. 33D. Unpaired Student's t-test between CMML vs. healthy control used to determine statistical significance, where P<0.05 was statistically significant. *P<0.05, **P<0.01, ***P<0.001, ****P<0.0001. [MFI mean fluorescence intensity]

FIGS. 34A-34 show CBL mutations hotspots in CMML cluster in the RING domain, unlike in JMML where they more commonly occur within the LHR. FIG. 34A shows a lollipop representation of mutation hotspots in key Cbl functional domains; contrasting frequencies detected in CMML (top) and JMML (bottom). Mutations detected in patients in the PREACH-M study are highlighted in red. FIG. 34B shows contingency table of CBL mutation hotspots within the LHR and RING domain of the protein. Data sourced from COSMIC and the PREACH-M study. FIG. 34C shows tertiary protein structure of native, inactive Cbl wildtype (PDB 2Y1M). The TKBD is colored beige, LHR blue and RING domain red. Amino acid residues of the top 6 mutation hotspots are indicated in green; Tyrosine371 (Y371), Leucine380 (L380), Cysteine384 (C384), Cysteine396 (C396), Cysteine404 (C404) and Arginine420 (R420). FIG. 34D shows X-ray structures of wildtype Cbl in unphosphorylated, inactive state and in closed conformation (PDB 2Y1M) (top left); wildtype Cbl in Y371 phosphorylated, active state and in open conformation (PDB 4A4C) (bottom left); mutant Cbl Y371E (PDB 5HKX) (top right) and mutant Cbl Y371F (PDB 5J3X) (bottom right). The TKBD is colored beige, LHR of wildtype blue, LHR of mutant cyan, RING domain of wildtype red, RING domain of mutant pink. Statistical analysis was performed using two-sided Fisher's exact test, where P<0.05 was statistically significant. ****P<0.0001 [TKBD=tyrosine kinase binding domain; LHR=linker helix region; RING=RING domain; UBA=ubiquitin-association domain; WT=wildtype; MUT=mutant; RMSD=root mean square deviation (distanced-based measure of protein structure similarity)]

FIGS. 35A-35B show cytokine receptor CD114, CD115, CD116 and CD131 expression by percentage positive cells (FIG. 35A) and mean fluorescence intensity (MFI) (FIG. 35B). The expression of CD114, CD115, CD116 and CD131 in CMML samples (n=4-6) vs control (n=2-3) in CD45+, CD34+ and CD14+ subpopulations. CBL mutant CMML represented by red squares, wildtype by clear squares. Bars represent medians. Unpaired t-test was applied for statistical analysis, where P<0.05 was considered significant. *P<0.05, **P<0.01, ***P<0.001, ****P<0.0001.

### DETAILED DESCRIPTION OF THE INVENTION

The present subject matter may be understood more readily by reference to the following detailed description which forms a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

As employed above and throughout the disclosure, the following terms and abbreviations, unless otherwise indicated, shall be understood to have the following meanings.

In the present disclosure the singular forms “a,” “an,” and “the” include the plural reference, and reference to a particular numerical value includes at least that particular value, unless the context clearly indicates otherwise. Thus, for example, a reference to “a compound” is a reference to one or more of such compounds and equivalents thereof known to those skilled in the art, and so forth. The term “plurality”, as used herein, means more than one. When a range of values is expressed, another embodiment incudes from the one particular and/or to the other particular value. Similarly, when values are expressed as approximations, by use of the antecedent “about,” it is understood that the particular value forms another embodiment. All ranges are inclusive and combinable.

As used herein, the terms “component,” “composition,” “composition of compounds,” “compound,” “drug,” “pharmacologically active agent,” “active agent,” “therapeutic,” “therapy,” “treatment,” or “medicament” are used interchangeably herein to refer to a compound or compounds or composition of matter which, when administered to a subject (human or animal) induces a desired pharmacological and/or physiologic effect by local and/or systemic action.

As used herein, the terms “treatment” or “therapy” (as well as different forms thereof) include preventative (e.g., prophylactic), curative or palliative treatment. As used herein, the term “treating” includes alleviating or reducing at least one adverse or negative effect or symptom of a condition, disease or disorder.

The terms “subject,” “individual,” and “patient” are used interchangeably herein, and refer to an animal, for example a human, to whom treatment for CMML, including prophylactic treatment, with the pharmaceutical compositions according to the present invention, i.e., the anti-hGM-CSF antibodies and the hypomethylating agents, respectively, is provided. The term “subject” as used herein refers to human and non-human animals. The terms “non-human animals” and “non-human mammals” are used interchangeably herein and include all vertebrates, e.g., mammals, such as non-human primates, (particularly higher primates), sheep, dog, rodent, (e.g., mouse or rat), guinea pig, goat, pig, cat, rabbits, cows, horses and non-mammals such as reptiles, amphibians, chickens, and turkeys.

CMML is a rare disease, which occurs in about four of every one million people in the U.S. each year; about 1,100 new cases of CMML are diagnosed annually. About nine out of ten cases are found in people 60 years of age and older. CMML occurs more often in men than in women and is very rare in young people.

CMML is a clonal stem cell disorder with features of both myelodysplasia and myeloproliferative disease; monocytes in the bone marrow begin to grow out of control, filling the bone marrow and preventing other blood cells from growing.

It is thought that Clonal Hematopoiesis of Indeterminate Potential (CHIP)/clonal hematopoiesis (CH) may give rise to CMML. While the exact cause of CMML is unknown, there are some known risk factors that increase the chances of getting CMML, including older age (60 or older), being male, being exposed to certain chemicals at work or in the environment, being exposed to radiation and past treatment with certain anticancer drugs.

Diagnosis of CMML includes persistent monocytosis>1×10⁹/L with an acquired clonal abnormality (excluding PDGFR/FGFR1/BCRABL/JAK2, ET/PRV/MF) and >94% CD14⁺CD16⁻ classic monocytes are pathonognomic of CMML. 60% of CMML patients have aTET2 mutation and 40% have a NRAS/KRAS/CBL mutation. The median overall survival after CMML diagnosis is 30 months.

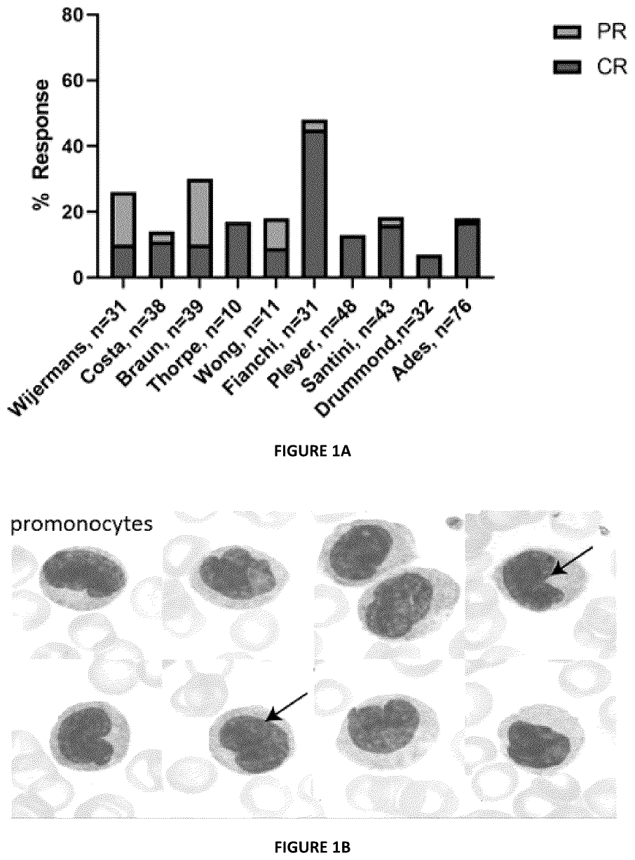

At present, azacitidine and decitabine, the only approved treatments for a select population of CMML patients leads to response rates (CR+PR) of 7-21% with limited survival benefit. FIG. 1A shows a combination of ten studies upon which the CR+PR rate for azacitidine alone in CMML of 7-21% is based.

To improve survival and progression-free survival of CMML patients after treatment compared to survival and progression-free survival after administration of a hypomethylating agent alone, which is the current SOC, a clinical trial for the treatment of CMML with combination therapy of anti-hGM-CSF antibody lenzilumab and azacitidine was commenced, as described in Example 1.

In one aspect, provided herein is a method for treating a subject having chronic myelomonocytic leukemia (CMML), the method comprising: (a) identifying a RAS pathway mutation in tumor cells of the subject, wherein the RAS pathway mutation is a NRAS, KRAS, PTPN-11 and/or CBL mutation; (b) identifying a dominant CBL mutation of CBL variant allele frequency of from <5% to >10%; and (c) administering to the subject identified in steps (a) and (b) or solely in step (b) a therapeutically effective amount of an anti-hGM-CSF antibody. In an embodiment of the herein provided method, the method further comprises administering a therapeutically effective amount of a second therapeutic agent. In some embodiments, the second therapeutic agent is a hypomethylating agent. In certain embodiments, the second therapeutic agent is a chemotherapy drug. In a particular embodiment, the chemotherapy drug is hydroxyurea. In particular embodiments, the hydroxyurea is administered orally as a capsule or tablet in a dose based on body weight of the subject. In an embodiment, the dose of hydroxyurea for an adult subject is 15 milligrams (mg) per kilogram (kg) of body weight per day, taken as a single dose; however, a physician may adjust dose, as needed. The administration and dose of hydroxyurea for a child subject is determined by the physician. In an embodiment of the herein provided method, the method further comprises administering the therapeutically effective amount of a hypomethylating agent for five to seven days starting on day one of administration of the anti-hGM-CSF antibody. In some embodiments, the methods further comprise identifying an increased percentage of CD116 and CD131 in CD34⁺ stem and progenitor cells in the subject compared to a healthy subject and/or further comprise identifying an increased percentage of CD14⁺ cells in the subject compared to a healthy subject. In particular embodiments, the hypomethylating agent is selected from the group consisting of azacytidine, decitabine, and a combination of decitabine and cedazuridine. In a particular embodiment of the herein provided methods, the anti-hGM-CSF antibody is lenzilumab. In some embodiments of said methods, the anti-hGM-CSF antibody is selected from the group consisting of Namilumab, Otilimab, Gimsilumab, and TJM2 (TJ003234). In an embodiment, the hypomethylating agent is selected from the group consisting of azacytidine, decitabine, and a combination of decitabine and cedazuridine and the anti-hGM-CSF antibody is lenzilumab. In some embodiments, the anti-hGM-CSF antibody is selected from the group consisting of consisting of Namilumab, Otilimab, Gimsilumab, and TJM2 (TJ003234). In an embodiment of the herein provided methods, the anti-hGM-CSF antibody lenzilumab is administered on day one and day 15 of cycle 1 and on day one only for all subsequent cycles. In certain embodiments of said methods, the all subsequent cycles consist of a total of 24 cycles, and each cycle consists of 28 days. In an embodiment of the herein provided methods, the subject has a RAS pathway mutation and a TET2 mutation identified in the tumor cells. In certain embodiments of said methods, the subject has a RAS pathway mutation and two TET2 mutation variants identified in the tumor cells. In some embodiments of said methods, the anti-hGM-CSF antibody lenzilumab is administered intravenously (IV) at a dose of from 552 mg to 1656 mg, wherein the dose of 552 mg is administered over a 1 hour IV infusion and the dose of 1656 mg is administered over 2 hour(s) IV infusion. In an embodiment, the anti-hGM-CSF antibody lenzilumab is administered at a dose of 552 mg over a 1 hour IV infusion. In some embodiments, the hypomethylating agent is administered subcutaneously at a dose of 75 mg/m². In an embodiment of said methods, the administration of the therapeutically effective amount of the anti-hGM-CSF antibody and the therapeutically effective amount of a hypomethylating agent demonstrates a complete response (CR) plus partial response (PR) of 50-90% compared to complete response (CR) plus partial response (PR) of 7-21% achieved with sole administration of a hypomethylating agent. In some embodiments, the response is a complete response or a partial response during 6 first cycles. In an embodiment of the herein provided methods, the CR or PR result in improved survival and progression-free survival at two years after treatment compared to survival and progression-free survival at two years after administration of a hypomethylating agent alone. In an embodiment of said methods, the said combined administration demonstrates/achieves clinical benefit at any point during 24 cycles. In certain embodiments, the clinical benefit comprises impact on physical and functional capacity of the subject, social well-being of the subject, hematological and non-hematologic safety and combinations thereof. In an embodiment, the hematological safety comprises (a) a decrease in C-reactive protein (CRP) by at least 50% within six months of administering the therapeutically effective amount of the therapeutically effective amount of the anti-hGM-CSF antibody and the hypomethylating agent compared to baseline CRP prior to treatment, and (b) an improvement in hematological parameters. In an embodiment, the hematological safety comprises (a) an improved bone marrow response of less than 5% blasts within 12 months or (b) a complete response in subjects having a medium increase in GM-CSF and pro-inflammatory cytokines found in an innate immune response and M1 macrophage activation compared to levels of GM-CSF and pro-inflammatory cytokines and M1 macrophage activation in healthy subjects within 12 months. In some embodiments, the clinical benefit comprising impact on the physical capacity of the subject comprises a reduction in splenomegaly. In certain embodiments, the hematological safety comprises a decrease in a variant allele frequency (VAF) of at least one identified RAS-pathway mutation, wherein the RAS-pathway mutation is a KRAS and/or CBL mutation. In an embodiment of the herein provided methods, the methods further comprise treating the subject with an allogeneic transplant. In another aspect, provided herein is a method for treating a subject having chronic myelomonocytic leukemia (CMML), the method comprising: (a) identifying a RAS pathway mutation in tumor cells of the subject, wherein the RAS pathway mutation is a NRAS, KRAS, PTPN-11 and/or CBL mutation; (b) identifying a dominant CBL mutation of CBL variant allele frequency of from <5% to >10%; and (c) administering to the subject identified in steps (a) and (b) or solely in step (b) a therapeutically effective amount of an anti-hGM-CSF antibody and a therapeutically effective amount of a second therapeutic agent. In some embodiments, the second therapeutic agent is a hypomethylating agent. In certain embodiments, the second therapeutic agent is a chemotherapy drug. In a particular embodiment, the chemotherapy drug is hydroxyurea. In particular embodiments, the hydroxyurea is administered orally as a capsule or tablet in a dose based on body weight of the subject. In an embodiment, the dose of hydroxyurea for an adult subject is 15 milligrams (mg) per kilogram (kg) of body weight per day, taken as a single dose; however, a physician may adjust dose, as needed. The administration and dose of hydroxyurea for a child subject is determined by the physician. In an embodiment of the herein provided method, the hypomethylating agent is selected from the group consisting of azacytidine, decitabine, and a combination of decitabine and cedazuridine. In certain embodiments, the methods further comprise identifying an increased percentage of CD116 and CD131 in CD34⁺ stem and progenitor cells in the subject compared to a healthy subject and/or further comprise identifying an increased percentage of CD14⁺ cells in the subject compared to a healthy subject. In an embodiment, the hypomethylating agent is administered for five to seven days starting on day one of administration of the anti-hGM-CSF antibody. In a particular embodiment of the herein provided method, the anti-hGM-CSF antibody is lenzilumab. In some embodiments, the anti-hGM-CSF antibody is selected from the group consisting of Namilumab, Otilimab, Gimsilumab, and TJM2 (TJ003234). In an embodiment, the hypomethylating agent is selected from the group consisting of azacytidine, decitabine, and a combination of decitabine and cedazuridine and the anti-hGM-CSF antibody is lenzilumab. In some embodiments of said methods, the anti-hGM-CSF antibody is selected from the group consisting of Namilumab, Otilimab, Gimsilumab, and TJM2 (TJ003234). In an embodiment, the anti-hGM-CSF antibody lenzilumab is administered on day one and day 15 of cycle 1 and on day one only for all subsequent cycles. In some embodiments, the all subsequent cycles consist of a total of 24 cycles, and each cycle consists of 28 days. In certain embodiments, the subject has a RAS pathway mutation and a TET2 mutation identified in the tumor cells. In various embodiments, the subject has a RAS pathway mutation and two TET2 mutation variants are identified in the tumor cells. In an embodiment, the anti-hGM-CSF antibody lenzilumab is administered intravenously (IV) at a dose of from 552 mg to 1656 mg, wherein the dose of 552 mg is administered over a 1 hour IV infusion and the dose of 1656 mg is administered over 2 hours IV infusion. In a particular embodiment of the herein provided methods, the anti-hGM-CSF antibody lenzilumab is administered at a dose of 552 mg over a 1 hour IV infusion. In some embodiments, the azacitidine, decitabine, or the combination of decitabine and cedazuridine is administered subcutaneously at a dose of 75 mg/m². In an embodiment, the administration of the anti-hGM-CSF antibody lenzilumab and the hypomethylating agent demonstrates a complete response (CR) plus partial response (PR) of 50-90% compared to a CR+PR rate of 7-21% achieved with sole administration of a hypomethylating agent. In some embodiments, the response is a complete response or a partial response during 6 first cycles. In certain embodiments of said methods, the CR or PR result in improved survival and progression-free survival at two years after treatment compared to survival and progression-free survival at two years after administration of a hypomethylating agent alone. In particular embodiments of the herein provided methods, the administration demonstrates/achieves clinical benefit at any point during 24 cycles. In some embodiments, the clinical benefit comprises impact on physical and functional capacity of the subject; social well-being of the subject, hematological and non-hematologic safety and combinations thereof. In certain embodiments, the hematological safety comprises (a) a decrease in C-reactive protein (CRP) by at least 50% within six months of administering the therapeutically effective amount of the therapeutically effective amount of the anti-hGM-CSF antibody and the hypomethylating agent compared to baseline CRP prior to treatment, and (b) an improvement in hematological parameters. In an embodiment, the hematological safety comprises (a) an improved bone marrow response of less than 5% blasts within 12 months or (b) a complete response in subjects having a medium increase in GM-CSF and pro-inflammatory cytokines found in an innate immune response and M1 macrophage activation compared to levels of GM-CSF and pro-inflammatory cytokines and M1 macrophage activation in healthy subjects within 12 months. In some embodiments, the clinical benefit comprising impact on the physical capacity of the subject comprises a reduction in splenomegaly. In an embodiment, the hematological safety comprises a decrease in a variant allele frequency (VAF) of at least one identified RAS-pathway mutation, wherein the RAS-pathway mutation is a KRAS and/or CBL mutation. In various embodiments, the hematological safety comprises (a) a decrease in C-reactive protein (CRP) by at least 50% within six months of administering the therapeutically effective amount of the therapeutically effective amount of the anti-hGM-CSF antibody and the hypomethylating agent compared to baseline CRP prior to treatment, and (b) an improvement in hematological parameters. In an embodiment, the hematological safety comprises (a) an improved bone marrow response of less than 5% blasts within 12 months or (b) a complete response in subjects having a medium increase in GM-CSF and pro-inflammatory cytokines found in an innate immune response and M1 macrophage activation compared to levels of GM-CSF and pro-inflammatory cytokines and M1 macrophage activation in healthy subjects within 12 months. In some embodiments of said methods, the clinical benefit comprising impact on the physical capacity of the subject comprises a reduction in splenomegaly. In certain embodiments, the hematological safety comprises a decrease in a variant allele frequency (VAF) of at least one identified RAS-pathway mutation, wherein the RAS-pathway mutation is a KRAS and/or CBL mutation. In an embodiment of said methods, the methods further comprise treating the subject with an allogeneic transplant.

In one aspect, provided herein is a method for treating a subject having chronic myelomonocytic leukemia (CMML), the method comprising: (a) identifying a RAS pathway mutation in tumor cells of the subject, wherein the RAS pathway mutation is a NRAS, KRAS, PTPN-11 and/or CBL mutation; and (b) administering to the subject identified in step (a) a therapeutically effective amount of an anti-hGM-CSF antibody. In an embodiment, the anti-hGM-CSF antibody is lenzilumab. In some embodiments, the anti-hGM-CSF antibody is selected from the group consisting of Namilumab, Otilimab, Gimsilumab, and TJM2 (TJ003234). In an embodiment, the method further comprises administering a therapeutically effective amount of a hypomethylating agent for five to seven days starting on day one of administration of the anti-hGM-CSF antibody. In certain embodiments, the hypomethylating agent is selected from the group consisting of azacytidine, decitabine, and a combination of decitabine and cedazuridine (INQOVI®). In an embodiment of the herein provided methods further comprising administering a therapeutically effective amount of a hypomethylating agent, the anti-hGM-CSF antibody is lenzilumab. In certain embodiments of said methods, the anti-hGM-CSF antibody is selected from the group consisting of Namilumab, Otilimab, Gimsilumab, and TJM2 (TJ003234). In some embodiments, the anti-hGM-CSF antibody, lenzilumab is administered on day one and day 15 of cycle 1 and on day one only for all subsequent cycles. In certain embodiments, the all subsequent cycles consist of a total of 24 cycles, and each cycle consists of 28 days. In various embodiments, the anti-hGM-CSF antibody, Namilumab, Otilimab, Gimsilumab, or TJM2 (TJ003234) is administered on day one and day 15 of cycle 1 and on day one only for all subsequent cycles. In some embodiments, the all subsequent cycles consist of a total of 24 cycles, and each cycle consists of 28 days. In a particular embodiment, the subject has a RAS pathway mutation and a TET2 mutation. In some embodiments, the subject has a RAS pathway mutation and two TET2 mutation variants. In certain embodiments of the herein provided methods, the anti-hGM-CSF antibody lenzilumab is administered intravenously (IV) at a dose of from 552 mg to 1656 mg, wherein the dose of 552 mg is administered over a 1 hour IV infusion and the dose of 1656 mg is administered over 2 hour(s) IV infusion. In a particular embodiment, the anti-hGM-CSF antibody lenzilumab is administered at a dose of 552 mg over a 1 hour IV infusion. In an embodiment, the hypomethylating agent is administered subcutaneously at a dose of 75 mg/m². In certain embodiments, the hypomethylating agent is selected from the group consisting of azacytidine, decitabine, and a combination of decitabine and cedazuridine (INQOVI®). In a particular embodiment, the hypomethylating agent is azacitidine. In certain embodiments, the combination therapy, i.e., the administration of the anti-hGM-CSF antibody and the hypomethylating agent, demonstrates, i.e., achieves a complete response (CR) plus partial response (PR) of 50-90% compared to a CR+PR rate of 7-21% achieved with sole administration of a hypomethylating agent, e.g., azacytidine, decitabine, or a combination of decitabine and cedazuridine (INQOVI®). In a particular embodiment, the anti-hGM-CSF antibody is lenzilumab. In some embodiments of said combination therapy methods, the anti-hGM-CSF antibody is selected from the group consisting of Namilumab, Otilimab, Gimsilumab, and TJM2 (TJ003234). In an embodiment, the response to said combination therapy, i.e., the administration of the anti-hGM-CSF antibody and the hypomethylating agent is a complete response or a partial response during 6 first cycles. In a particular embodiment, the CR or PR result in improved survival and progression-free survival at two years after treatment compared to survival and progression-free survival at two years after administration of a hypomethylating agent alone. In certain embodiments, the administration of the combination therapy of the anti-hGM-CSF antibody and the hypomethylating agent demonstrates, that is, achieves, clinical benefit at any point during 24 cycles. In some embodiments of the herein provided methods, the clinical benefit comprises impact on physical and functional capacity of the subject; social well-being of the subject, hematological and non-hematologic safety and combinations thereof. In an embodiment of the provided methods, the hematological safety comprises (a) a decrease in C-reactive protein (CRP) by at least 50% within six months of administering the therapeutically effective amount of the therapeutically effective amount of the anti-hGM-CSF antibody and the hypomethylating agent compared to baseline CRP prior to treatment, and (b) an improvement in hematological parameters. In some embodiments, the hematological safety comprises (a) an improved bone marrow response of less than 5% blasts within 12 months or (b) a complete response in subjects having a medium increase in GM-CSF and pro-inflammatory cytokines found in an innate immune response and M1 macrophage activation compared to levels of GM-CSF and pro-inflammatory cytokines and M1 macrophage activation in healthy subjects within 12 months. In certain embodiments, the clinical benefit comprising impact on the physical capacity of the subject comprises a reduction in splenomegaly. In some embodiments, the hematological safety comprises a decrease in a variant allele frequency (VAF) of at least one identified RAS-pathway mutation, wherein the RAS-pathway mutation is a KRAS and/or CBL mutation. In a particular embodiment, the provided methods further comprise treating the subject with an allogeneic transplant.

In another aspect, provided herein is a method for treating a subject having chronic myelomonocytic leukemia (CMML), the method comprising: (a) identifying a RAS pathway mutation in tumor cells of the subject, wherein the RAS pathway mutation is a NRAS, KRAS, PTPN-11 and/or CBL mutation; and (b) administering to the subject identified in step (a) a therapeutically effective amount of an anti-hGM-CSF antibody and a therapeutically effective amount of a hypomethylating agent. In an embodiment, the anti-hGM-CSF antibody is lenzilumab. In some embodiments, the anti-hGM-CSF antibody is selected from the group consisting of Namilumab, Otilimab, Gimsilumab, and TJM2 (TJ003234). In some embodiments, the hypomethylating agent is selected from the group consisting of azacytidine, decitabine, and a combination of decitabine and cedazuridine (INQOVI®). In a particular embodiment, the hypomethylating agent is azacitidine. In certain embodiments, the hypomethylating agent is administered for five to seven days starting on day one of administration of the anti-hGM-CSF antibody. In some embodiments of said methods, the anti-hGM-CSF antibody is lenzilumab. In certain embodiments of said methods, the anti-hGM-CSF antibody is selected from the group consisting of Namilumab, Otilimab, Gimsilumab, and TJM2 (TJ003234). In a particular embodiment of the herein provided methods, the anti-hGM-CSF antibody lenzilumab is administered on day one and day 15 of cycle 1 and on day one only for all subsequent cycles. In an embodiment, the all subsequent cycles consist of a total of 24 cycles, and each cycle consists of 28 days. In some embodiments, the anti-hGM-CSF antibody elected from the group consisting of Namilumab, Otilimab, Gimsilumab, and TJM2 (TJ003234) is administered on day one and day 15 of cycle 1 and on day one only for all subsequent cycles. In some embodiments, the all subsequent cycles consist of a total of 24 cycles, and each cycle consists of 28 days. In particular embodiments, the subject has a RAS pathway mutation in tumor cells of the subject, and a TET2 mutation. In certain embodiments, the subject has a RAS pathway mutation and two TET2 mutation variants in tumor cells of the subject. In a particular embodiment, the anti-hGM-CSF antibody lenzilumab is administered intravenously (IV) at a dose of from 552 mg to 1656 mg, wherein the dose of 552 mg is administered over a 1 hour IV infusion and the dose of 1656 mg is administered over 2 hours IV infusion. In some embodiments, the anti-hGM-CSF antibody lenzilumab is administered at a dose of 552 mg over a 1 hour IV infusion. In an embodiment, the azacitidine, decitabine, or the combination of decitabine and cedazuridine is administered subcutaneously at a dose of 75 mg/m². In certain embodiments, the combination therapy, i.e., the administration of the anti-hGM-CSF antibody and the hypomethylating agent, demonstrates/achieves a complete response (CR) plus partial response (PR) of 50-90% compared to a CR+PR rate of 7-21% achieved with sole administration of a hypomethylating agent. In a particular embodiment, the anti-hGM-CSF antibody is lenzilumab. In some embodiments of said methods, the anti-hGM-CSF antibody is selected from the group consisting of Namilumab, Otilimab, Gimsilumab, and TJM2 (TJ003234). In an embodiment, the response to said combination therapy is a complete response or a partial response during 6 first cycles. In some embodiments, the CR or PR result in improved survival and progression-free survival at two years after treatment compared to survival and progression-free survival at two years after administration of a hypomethylating agent alone. In a particular embodiment, the administration demonstrates/achieves clinical benefit at any point during 24 cycles. In an embodiment, the clinical benefit comprises impact on physical and functional capacity of the subject; social well-being of the subject, hematological and non-hematologic safety and combinations thereof. In an embodiment of the provided methods, the hematological safety comprises (a) a decrease in C-reactive protein (CRP) by at least 50% within six months of administering the therapeutically effective amount of the therapeutically effective amount of the anti-hGM-CSF antibody and the hypomethylating agent compared to baseline CRP prior to treatment, and (b) an improvement in hematological parameters. In certain embodiments, the hematological safety comprises (a) an improved bone marrow response of less than 5% blasts within 12 months or (b) a complete response in subjects having a medium increase in GM-CSF and pro-inflammatory cytokines found in an innate immune response and M1 macrophage activation compared to levels of GM-CSF and pro-inflammatory cytokines and M1 macrophage activation in healthy subjects within 12 months. In some embodiments, the clinical benefit comprising impact on the physical capacity of the subject comprises a reduction in splenomegaly. In an embodiment, the hematological safety comprises a decrease in a variant allele frequency (VAF) of at least one identified RAS-pathway mutation, wherein the RAS-pathway mutation is a KRAS and/or CBL mutation. In certain embodiments, the provided methods further comprise treating the subject with an allogeneic transplant.

DNA sequencing is performed by whole genome sequencing or whole exome sequencing (targeted sequencing) techniques detect the RAS (NRAS, KRAS, CBL) and/or TET2 gene mutations. Both blood and bone marrow may be used for screening to detect bone marrow blasts, peripheral blood cell counts, and mutational status.

The CMML patients in Example 1 are relapsed refractory CMML patients.

### Lenzilumab

Lenzilumab (also referred to herein as “LENZ”) is a proprietary Humaneered® first-in-class monoclonal antibody with best-in-class specificity and affinity that neutralizes human GM-CSF (hGM-CSF) by preventing binding to and hence signaling through its receptor, as described in U.S. Pat. Nos. 8,168,183 and 9,017,674, each of which is incorporated herein by reference in its entirety.

LENZ is a recombinant monoclonal antibody, derived from mouse antibody LMM102, targeting hGM-CSF, with potential immunomodulatory activity, high binding affinity in the picomolar range, 94% homology to human germline, and has low immunogenicity. Following intravenous administration, lenzilumab binds to and neutralizes GM-CSF, preventing hGM-CSF binding to its receptor, thereby preventing hGM-CSF-mediated signaling to myeloid progenitor cells.

Lenzilumab comprises a VH region (VH #5) having the amino acid sequence: QVQLVQSGAEVKKPGASVKVSCKASGYSFTNYYIHWVRQAPGQRLEWMGWINAGNG NTKYSQKFQGRVTITRDTSASTAYMELSSLRSEDTAVYYCVRRQRFPYYFDYWGQGTL VTVSS (SEQ ID NO: 1) and a VL region (VK #2) having the amino acid sequence: EIVLTQSPATLSVSPGERATLSCRASQSVGTNVAWYQQKPGQAPRVLIYSTSSRATGITD RFSGSGSGTDFTLTISRLEPEDFAVYYCQQFNKSPLTFGGGTKVEIK (SEQ ID NO: 2).

### Azacitidine and Other Demethylating Agents

A hypomethylating agent (or demethyating agent) is a drug that inhibits DNA methylation, i.e., the modification of DNA nucleotides by addition of a methyl group. Azacitidine is an analog of the nucleoside cytidine that inhibits DNA methyltransferase, impairing DNA methylation. Azacitidine (also referred to herein as “AZA”) is a ribonucleoside, and thus incorporates primarily into RNA to a larger extent than into DNA. Azacitidine's antineoplastic activity comprises two mechanisms: (1) the inhibition of DNA methyltransferase at low doses, causing hypomethylation of DNA, and (2) direct cytotoxicity in abnormal hematopoietic cells in the bone marrow through its incorporation into DNA and RNA at high doses, resulting in cell death. Oral azacitidine (ONUREG) is used for continued treatment of adult patients with acute myeloid leukemia (AML), which achieved first complete remission (CR) or complete remission with incomplete blood count recovery (CRi) following intensive induction chemotherapy and are not able to complete intensive curative therapy.

The hypomethylating agent decitabine (5-aza-2′-deoxycytidine) is a deoxyribonucleoside that only incorporates into DNA. Decitabine is used to treat blood/bone marrow disorders (myelodysplastic syndromes; “MDS”). Decitabine/cedazuridine (INQOVI®) is a combination of decitabine and cedazuridine, a cytidine deaminase inhibitor. Decitabine/cedazuridine is used to treat adults with myelodysplastic syndromes (MDS), including CMML.

### Pharmaceutical Compositions

The anti-hGM-CSF antibodies and the hypomethylating agents described and administered according to the methods provided herein are formulated as pharmaceutical compositions comprising an anti-hGM-CSF antibody and a hypomethylating agent, respectively, and one or more pharmaceutically acceptable carriers. “Pharmaceutically acceptable carriers” include any excipient which is nontoxic to the cell or mammal being exposed thereto at the dosages and concentrations employed. The pharmaceutical composition may include one or more therapeutic agents.

Thus, as used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

In an embodiment, pharmaceutical compositions containing the therapeutic agent or agents described herein, can be, in one embodiment, administered to a subject by any method known to a person skilled in the art, such as, without limitation, orally, parenterally, transnasally, transmucosally, subcutaneously, transdermally, intramuscularly, intravenously, intraarterially, intra-dermally, intra-peritoneally, intra-ventricularly, intra-cranially, intra-vaginally, or intra-tumorally.

Carriers may be any of those conventionally used, as described above, and are limited only by chemical-physical considerations, such as solubility and lack of reactivity with the compound of the invention, and by the route of administration. The choice of carrier will be determined by the particular method used to administer the pharmaceutical composition. Some examples of suitable carriers include lactose, glucose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water and methylcellulose. The formulations can additionally include lubricating agents such as talc, magnesium stearate, and mineral oil; wetting agents, surfactants, emulsifying and suspending agents; preserving agents such as methyl- and propylhydroxybenzoates; sweetening agents; flavoring agents, colorants, buffering agents (e.g., acetates, citrates or phosphates), disintegrating agents, moistening agents, antibacterial agents, antioxidants (e.g., ascorbic acid or sodium bisulfite), chelating agents (e.g., ethylenediaminetetraacetic acid), and agents for the adjustment of tonicity such as sodium chloride. Other pharmaceutical carriers can be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. In one embodiment, water, preferably bacteriostatic water, is the carrier when the pharmaceutical composition is administered intravenously or intratumorally. Saline solutions and aqueous dextrose and glycerol solutions can also be employed as liquid carriers, particularly for injectable solutions.

Pharmaceutical compositions suitable for injectable use may include sterile aqueous solutions (where water soluble) or dispersions and sterile powders for the extemporaneous preparation of sterile injectable solutions or dispersion. For intravenous administration, suitable carriers include, without limitation, physiological saline, bacteriostatic water, Cremophor EL.TM. (BASF, Parsippany, N.J.) or phosphate buffered saline (PBS). The composition should be sterile and should be fluid to the extent that easy syringeability exists. It should be stable under the conditions of manufacture and storage and be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (anti-hGM-CSF antibodies and the hypomethylating agents, respectively) in the required amount to produce a therapeutic effect in an appropriate solvent with one or a combination of ingredients enumerated above, as appropriate, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

The pharmaceutical compositions and formulations comprising an anti-hGM-CSF antibody and the pharmaceutical compositions and formulations comprising a hypomethylating agent, as respectively described herein, may be administered alone or with other biologically-active agents. Administration can be systemic or local, e.g., through portal vein delivery to the liver. In addition, it may be advantageous to administer the composition into the central nervous system by any suitable route, including intraventricular and intrathecal injection. Intraventricular injection may be facilitated by an intraventricular catheter attached to a reservoir (e.g., an Ommaya reservoir). Pulmonary administration may also be employed by use of an inhaler or nebulizer, and formulation with an aerosolizing agent. It may also be desirable to administer the Therapeutic locally to the area in need of treatment; this may be achieved by, for example, and not by way of limitation, local infusion during surgery, topical application, by injection, by means of a catheter, by means of a suppository, or by means of an implant.

Moreover, “pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio. The term “pharmaceutically acceptable” also includes those carriers approved by a regulatory agency of the Federal or a state government or listed in the U.S. Pharmacopeia or other generally recognized pharmacopeia for use in animals and, more particularly, in humans.

### Effective Doses

Effective doses of the pharmaceutical compositions of the present invention, for treatment of conditions or diseases, such as CMML, vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages may be titrated using routine methods known to those of skill in the art to optimize safety and efficacy. The pharmaceutical compositions of the invention thus may include a “therapeutically effective amount.” A “therapeutically effective amount” refers to an amount effective, at dosages and for periods of time necessary, to achieve the desired therapeutic result. A therapeutically effective amount of a molecule may vary according to factors such as the disease state, age, sex, and weight of the individual, and the ability of the molecule to elicit a desired response in the individual. A therapeutically effective amount is also one in which any toxic or detrimental effects of the molecule are outweighed by the therapeutically beneficial effects.

Furthermore, a skilled artisan would appreciate that the term “therapeutically effective amount” may encompass total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

The amount of a compound of the invention that will be effective in the treatment of a particular disorder or condition, in particular CMML, also will depend on the nature of the disorder or condition, and can be determined by standard clinical techniques. In addition, in vitro assays may optionally be employed to help identify optimal dosage ranges. The precise dose to be employed in the formulation will also depend on the route of administration, and the seriousness of the disease or disorder, and should be decided according to the judgment of the practitioner and each patient's circumstances. In one embodiment, the pharmaceutical composition comprising an anti-hGM-CSF antibody is administered intravenously at a dose of from 552 mg to 1656 mg, wherein the dose of 552 mg is administered over a 1 hour IV infusion. In some embodiments, the pharmaceutical composition comprising an anti-hGM-CSF antibody is administered intravenously at a dose of 1656 mg over 2 hour(s) IV infusion. In a particular embodiment, the pharmaceutical composition comprises the anti-hGM-CSF antibody lenzilumab. In some embodiments, the pharmaceutical composition comprises an anti-hGM-CSF antibody selected from the group consisting of Namilumab, Otilimab, Gimsilumab, and TJM2 (TJ003234).

The hypomethylating agent is administered subcutaneously at a dose of 75 mg/m². In certain embodiments of the pharmaceutical composition comprising a hypomethylating agent, the hypomethylating agent is selected from the group consisting of azacytidine, decitabine, and a combination of decitabine and cedazuridine.

The compound(s) or composition(s) of the invention may be administered only once, or it may be administered multiple times. For multiple dosages, the composition may be, for example, administered three times a day, twice a day, once a day, once every two days, twice a week, weekly, once every two weeks, or monthly.

The following examples are presented in order to more fully illustrate the preferred embodiments of the invention. They should in no way be construed, however, as limiting the broad scope of the invention.

### Example 1

Treatment of CMML with Anti-GM-CSF Antibody Lenzilumab and Azacitidine

The PREcision Approach to CHronic Myelomonocytic Leukemia (PREACH-M) trial assesses the efficacy of LENZ in CMML (ACTRN12621000223831p) to improve outcomes beyond those afforded by SOC.

### Methods

PREACH-M is a Phase 2/3 non-randomized, open-label precision medicine trial in 72 adults aged at least 18 years, newly diagnosed with WHO 2016 criteria for CMML; cytopenia (hemoglobin<100 g/L, platelets<100×10⁹/L or absolute neutrophil count<1.8×10⁹/L): white cell count≥13×10⁹/L; as well as TET2 (tet methylcytosine dioxygenase 2) and/or RAS pathway mutations (NRAS, KRAS, CBL).

Key exclusion criteria include prior treatment with investigational agents; radiotherapy within 28 days before treatment; treatment with G-CSF within 7 days of screening; GM-CSF within 28 days of screening; and uncontrolled medical conditions.

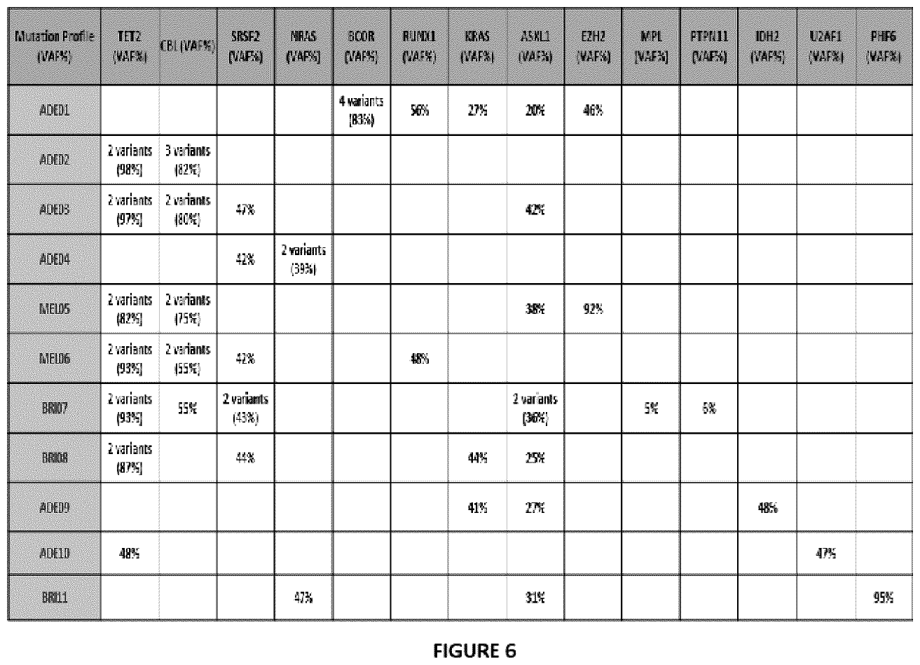

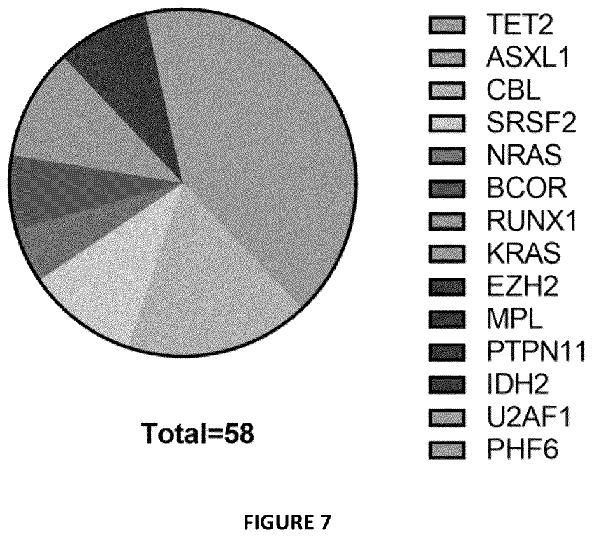

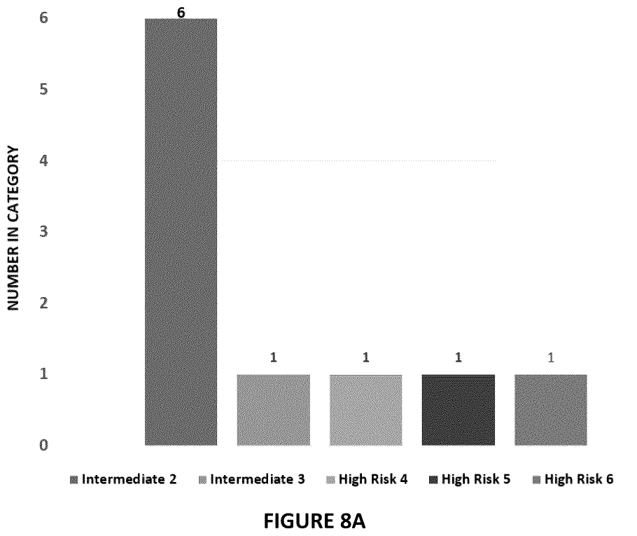

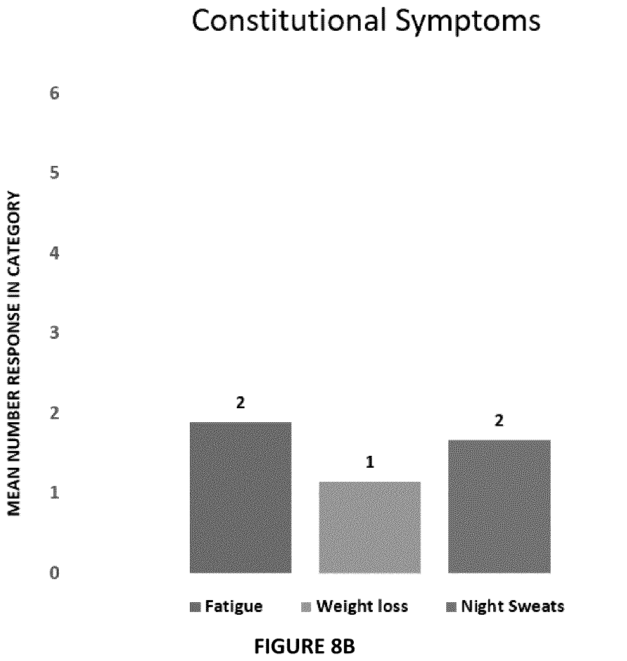

Subjects exhibiting RAS pathway mutations in their tumor cells, with or without TET2 mutations, receive 24 cycles (28 days) of azacitidine (SC; 75 mg/m² for 7 days) and LENZ (IV; 552 mg; d1 & d15 of cycle 1 and d1 only for all subsequent cycles); while those with non-RAS pathway mutations in their tumor cells, receive the same azacitidine regimen and sodium ascorbate (IV; 30 g for 7 days [15 g for 1st dose only, 30 g thereafter if no evidence of tumor lysis syndrome]; PO; 1.1 g on all other days). FIG. 6 shows the gene mutation profile baseline characteristics of 11 subjects in the lenzilumab cohort, including NRAS, KRAS, and/or CBL mutations and TET2 mutations. FIG. 7 shows in a pie chart format the genes mutated in the lenzilumab cohort. Cbl mutations are frequent, as are TET2 mutations.

Subjects who complete 24 cycles of treatment are followed every 6 months for 24 months for survival, disease status, and CMML-related therapy.

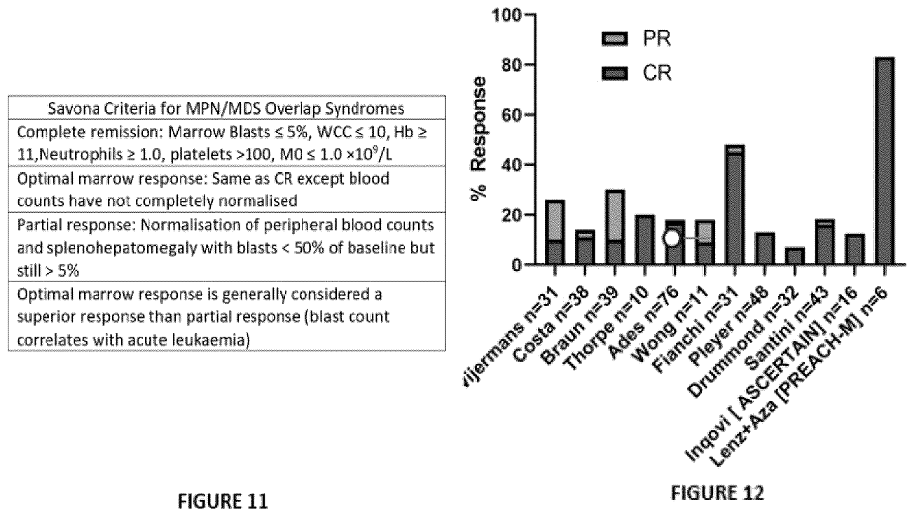

The primary endpoint is the frequency of CR or PR at any time during the first 12 cycles according to Savona Criteria (FIG. 11). Secondary endpoints include overall survival and progression-free survival at 2 years; proportion of subjects with clinical benefit at any point during the 24 cycles; impact on physical and functional capacity; social well-being according to Multidimensional Geriatric Assessment and quality of life; as well as hematological and non-hematologic safety.

### Results

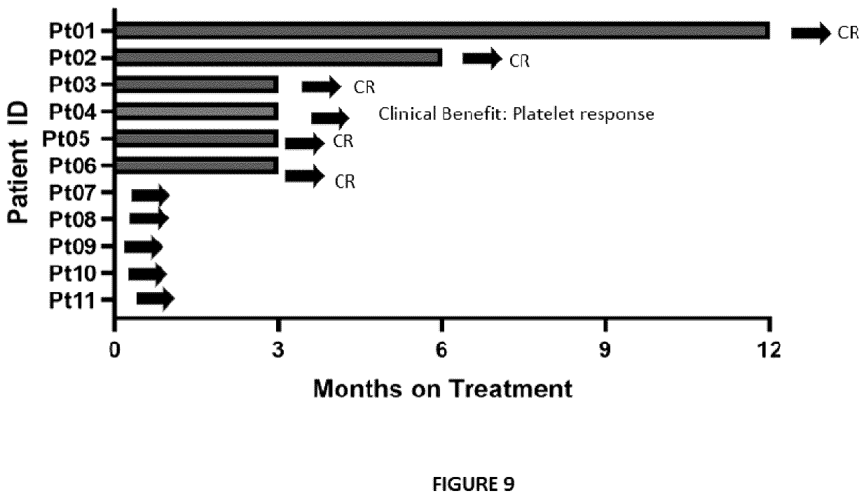

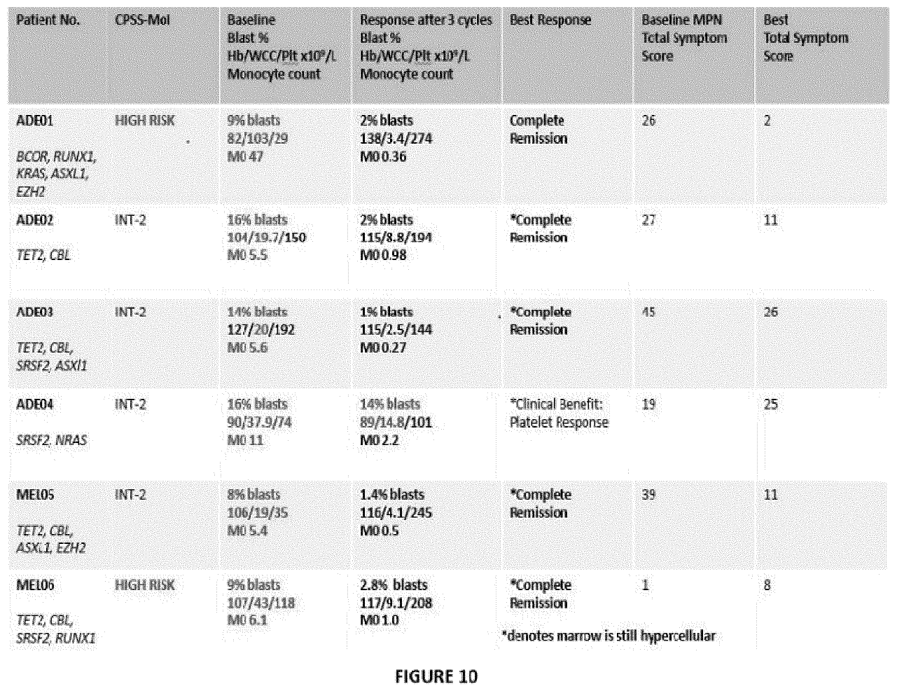

As of Dec. 31, 2022, eleven subjects were treated with azacitidine and LENZ (5 females, mean age of 67 years; 3 males, mean age of 69 years); among them 6 were evaluable based on at least 3 months of follow-up. Complete response (CR) or objective responses were observed in all evaluable patients including 2 with high risk based on molecular profiling (FIG. 10).

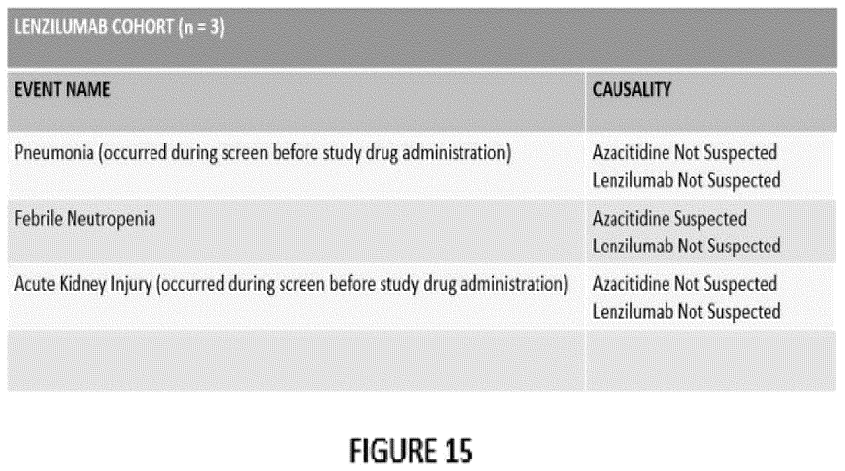

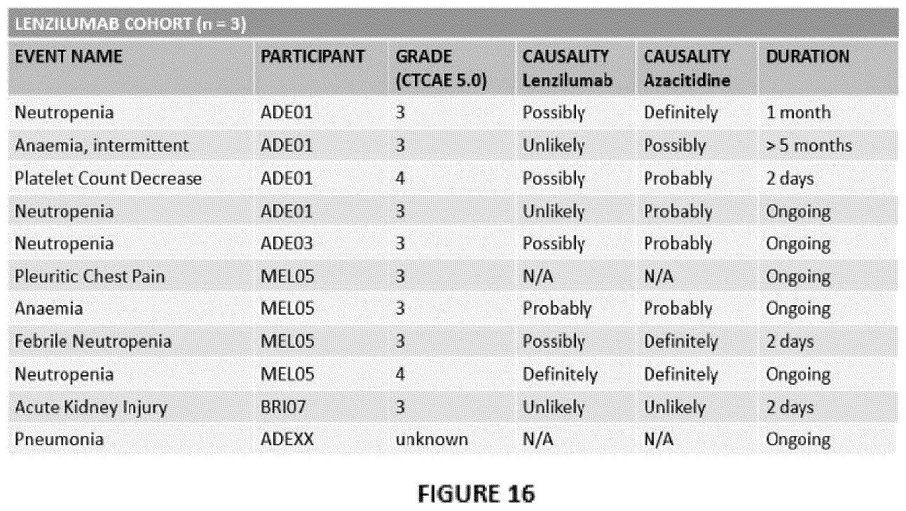

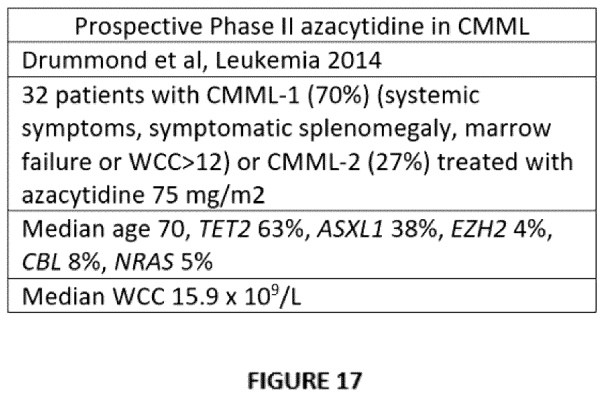

10 grade 3/4 Serious Adverse Events (AE) were observed of which 2 were assessed by the investigator as possibly related to LENZ. (FIGS. 15 and 16) Regarding the adverse effects summarized in FIG. 16, the investigators made a judgement regarding whether, in their opinion, the adverse event was related to a study drug. Options will include:

Definitely: The investigator feels that there is compelling evidence that there is a direct causal relationship between the AE and administration of the study drug.

Probably: there is evidence to suggest a causal relationship, and the influence of other causalities is unlikely.

Possibly: there is some evidence to suggest a causal relationship (i.e., there is a reasonable possibility that the AE may have been caused by the study drug). However, the role of other factors may have contributed to the event or it is possible that other factors may have been responsible for the event.

Unlikely: there is little evidence to suggest that there is a causal relationship with a study drug and there is another reasonable explanation for the event.

Not applicable: the participant did not receive study drug.

AZA is known to cause neutropenia, but lenzilumab has never been associated with neutropenia in any other studies. This is the first time that anti-hGM-CSF antibody, LENZ, and AZA have been used together in human subjects. Although LENZ does not cause neutropenia when dosed alone it is possible that AZA+LENZ could cause neutropenia.

### Conclusion

The ongoing PREACH-M trial evaluated GM-CSF neutralization with LENZ in addition to standard of care (AZA), in the treatment of patients having CMML with RAS pathway mutations or RAS and TET2 mutations in their tumor cells.

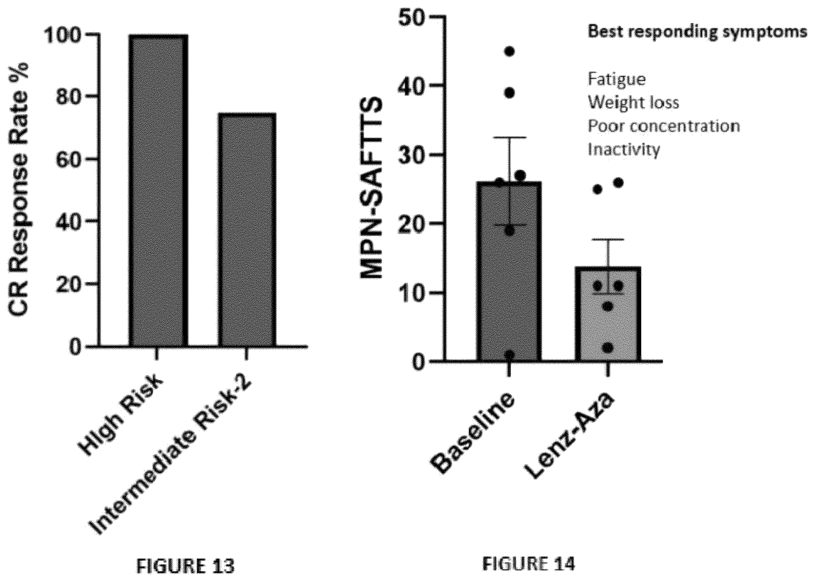

A superior early response data of Lenz+Aza combination therapy for CMML was shown compared to published literature for CMML treatment with DNA methyltransferase inhibitors (DNMTi) alone. (FIG. 12) A high CR response rate (%) obtained from Lenz+Aza combination therapy in high risk CMML patients was also shown. (FIG. 13). Likewise, a robust improvement in quality of life based on the MPN symptoms assessment form: total symptom score compared to baseline scores was demonstrated, of which the best responding symptoms were fatigue, weight loss, poor concentration, and inactivity.

### Example 2

### Lenzilumab in Addition to Azacitidine Improves Complete Response Rates in Chronic Myelomonocytic Leukemia

Chronic myelomonocytic leukemia (CMML) is a rare cancer orchestrated by granulocyte-macrophage colony-stimulating factor (GM-CSF), a pro-inflammatory cytokine that drives leukemic monocyte proliferation. Standard of care (SOC) for CMML treatment includes azacitidine (AZA), with a complete response (CR) rate of 16-21% (as described by Xu Y, et al., Real-world data on efficacy and safety of azacitidine therapy in chronic myelomonocytic leukemia in China: results from a multicenter, retrospective study. Invest New Drugs 2022;40(5):1117-1124. DOI:10.1007/s10637-022-01283-x, and Zheng X, et al., Efficacy and Safety of Hypomethylating Agents in Chronic Myelomonocytic Leukemia: A Single-Arm Meta-analysis. Glob Med Genet 2022;9(2):141-151. DOI: 10.1055/s-0042-1744157, each of which is incorporated herein by reference in its entirety).

The PREcision Approach to Chronic Myelomonocytic Leukemia (PREACH-M; ACTRN12621000223831) trial investigates novel CMML therapies directed by molecular profiling. Lenzilumab (LENZ; Humanigen, Inc., Short Hills, NJ) a proprietary Humaneered® first-in-class monoclonal antibody with best-in-class off-rate and affinity that neutralizes GM-CSF. PREACH-M interim results show that LENZ/AZA improves hematologic parameters, decreases spleen size, and dampens pro-inflammatory responses in CMML with RAS-pathway mutations. This report details the objective clinical responses from an interim analysis of the first 11 subjects who completed at least three months LENZ/AZA treatment.

### Methods

PREACH-M is a Phase 2/3 nonrandomized, uncontrolled, open-label trial in 72 adults aged at least 18 years, newly diagnosed with WHO 2016 criteria for CMML. Key exclusion criteria include prior treatment with investigational agents; radiotherapy within 28 days before treatment; treatment with G-CSF within 7 days of screening; GM-CSF within 28 days of screening; and uncontrolled medical conditions. Subjects exhibiting RAS pathway mutations (NRAS, KRAS, CBL) receive 24 cycles (every 28 days) of AZA (SC; 75 mg/m² for 7 days) and LENZ (IV; 552 mg; d1 & d15 of cycle 1 and d1 only for all subsequent cycles); while those with only TET2 mutations receive the same AZA regimen and sodium ascorbate (IV; 30 g for 7 days [15 g for 1st dose only, 30 g thereafter if no evidence of tumor lysis syndrome]; PO; 1.1 g on all other days). Subjects who complete 24 cycles of treatment are followed every 6 months for an additional 24 months. The primary endpoint is the frequency of complete response (CR) or partial response (PR) during the first 12 cycles according to Savona Criteria. Secondary endpoints include responses according to modified 2006 International Working Group criteria, 2 year overall survival, and symptom improvement.

### Results

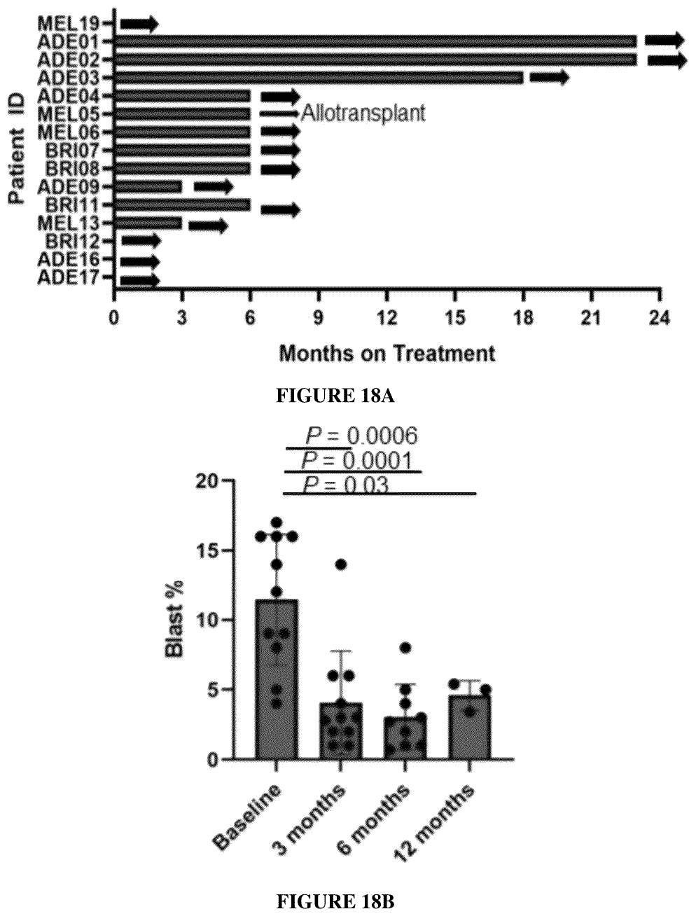

As of July 2023, 15 subjects were enrolled in the LENZ/AZA arm (8 females, 7 males with mean age 69; mean white cell count 21×10⁹/L, mean Hb 121 g/L; mean platelet count, 74×10⁹/L, mean blast count, 10.1%). (FIGS. 18A-18B) Mutations included; CBL (47% of subjects), NRAS (27%), KRAS (47%), NRAS and KRAS (13%), and TET2 (93%). Subjects exhibited CPSS-MOL scores, of intermediate risk 1 (n=1), intermediate risk 2-3 (n=8), and high risk 4-6 (n=6). All the 11 evaluable subjects at 3 months responded to LENZ/AZA. (FIG. 18A) CR was achieved within 3 cycles in 55% of subjects. Six of the subjects demonstrated CR including 2 with a high risk CPSS-MOL profile and 8 achieved either CR or complete marrow response (blasts<5%) within 12 months. (FIG. 18B) One subject had a platelet response, 1 subject each had PR and stable disease with blasts <5%. CMML progression was absent and 1 subject became eligible for allogeneic transplant. These findings exceed historical CR rates for hypomethylating agents (16%; 95% CI, 12-21% (as described by Xu Y, et al., 2022 supra) and 21%; 13-29% (as described by Zheng X, et al., 2022 supra)). Self-reported symptom scores from the standardized MPN-SAFFS improved from baseline (mean of 22 vs 12, P=0.06). Fifteen grade 3 and 9 grade 4 adverse events were reported of which 2 were “probably” ascribed to both LENZ/AZA and 7 were “possibly” ascribed to LENZ. No unexpected adverse events were observed.

### Conclusion

Interim analysis of the PREACH-M trial demonstrated that GM-CSF neutralization with LENZ/AZA, for the treatment of CMML with RAS-pathway mutations resulted in 55% CR, achieved early in treatment, durability up to 18 months, thus far, and no unexpected serious adverse events. These data suggest CMML is driven by a non-redundant cytokine that responds to immunotherapy.

### Example 3

### Suppression of KRAS and CBL Mutations and Hematological Improvement by Lenzilumab and Azacitidine Treatment in Proliferative Chronic Myelomonocytic Leukemia

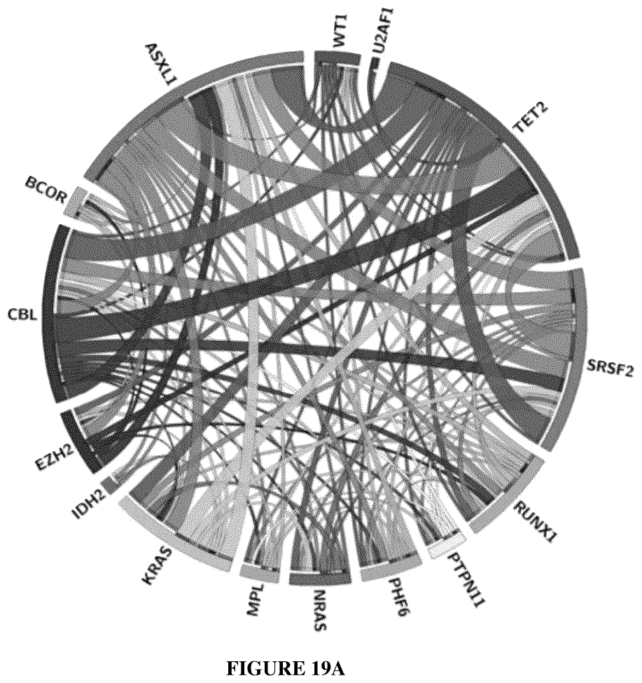

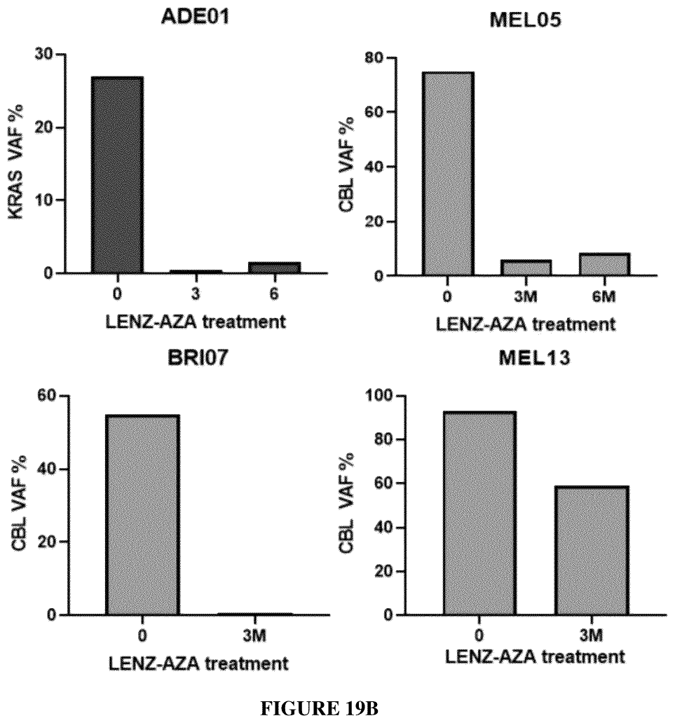

Mutations in RAS-pathway genes (NRAS, KRAS, CBL) are amongst the most common somatic mutations in cancer and historically resistant to most therapies. (FIG. 19A) Targeted therapies that impact the RAS-pathway constitute a major unmet need in oncology. The proliferative form of chronic myelomonocytic leukemia (CMML) is commonly associated with RAS-pathway mutations and has a high propensity to develop into acute myeloid leukemia. In pre-clinical models, Lenzilumab (LENZ; Humanigen, Inc., Short Hills, NJ), a proprietary Humaneered® first-in-class monoclonal antibody with best-in-class off-rate and affinity that neutralizes GM-CSF, resulted in a reduction of colony numbers and viability of CMML cells, with the greatest sensitivity in cells possessing RAS-pathway (NRAS/KRAS/CBL) mutations, suggesting benefit in the targeted treatment of CMML. (FIG. 19B) The PREcision Approach to CHronic Myelomonocytic Leukemia (PREACH-M) trial assesses the efficacy of LENZ, in addition to azacytidine (AZA), in CMML subjects with RAS-pathway mutations and high dose sodium ascorbate (ASC) and AZA in CMML subjects without RAS-pathway mutations. Interim data from 11 subjects with RAS-pathway (NRAS/KRAS/CBL) mutations in PREACH-M receiving LENZ/AZA demonstrate reductions in circulating GM-CSF and CRP with 8 subjects achieving complete response or optimal marrow response. This Example describes improvements in variant allele frequencies (VAF) for RAS-pathway mutations (FIG. 19B) and hematologic improvements associated with LENZ/AZA treatment in CMML.

### Methods

PREACH-M is a Phase 2/3 non-randomized, open-label trial in 72 subjects, aged at least 18 years, with newly diagnosed CMML based on the WHO 2016 criteria; and RAS-pathway mutations at a variant allele frequency ≥3%. Subjects received 24 cycles (every 28 days) of AZA (SC; 75 mg/m² for 7 days) and LENZ (IV; 552 mg; d1 & d15 of cycle 1 and d1 only for all subsequent cycles). Subjects without RAS-pathway mutations received the same AZA regimen and sodium ascorbate (IV; 30 g for 7 days (15 g for 1st dose only; 30 g thereafter if no evidence of tumor lysis syndrome); PO; 1.1 g on all other days). VAF was determined from bone marrow mononuclear cells using a 41-myeloid panel using Illumina Hi-Seq with a depth of 1000× performed on bone marrow aspirates obtained at baseline and Day 1 of treatment cycles 4, 7, and 12.

### Results

As of July 2023, 11 subjects completed at least 3 months of LENZ/AZA treatments and follow-up sequencing data were available for 10 subjects. Five of 10 subjects showed >10% decrease in VAF for at least one mutation detected at baseline, including 5 decreased alleles in the RAS-pathway (KRAS, CBL) out of 22 total VAF mutation responses. (FIG. 19B) CBL mutations showed the largest change with a decrease in >50% VAF in three subjects. (FIG. 19B) Decreases in RAS-pathway clones by LENZ/AZA were durable at 6 months. No decreases in VAF for any mutations were observed in 2 evaluable subjects following ASC/AZA treatment. Following 3 months LENZ/AZA treatment, blood monocyte count improved from a baseline of 11.0±6.0×10⁹/L to 2.0±1.0×10⁹/L (p=0.030). Blast differential improved from 10.5±5.0% to 4.0±3.5% (p=0.038). Platelet count increased from 90.0±50.0×10⁹/L to 150.0±60.0×10⁹/L (p=0.010). Statistical improvements remained durable for at least 6 (n=6) and 12 (n=3) months. Hemoglobin concentration increased from 100.0±22.0 g/L to 115.0±10.0 g/L at 3 months (p=NS, n=11) and progressively increased to 122.0±10.0 g/L at 12 months (p=0.024, n=3). Spleen length decreased from 15.0±4.0 cm to 13.0±4.0 cm (p=0.03) and decreased further to 11.0±2.0 cm in subjects who received 12 months of treatment (n=3). No evidence of disease progression or relapse was observed in any subject.

### Conclusion

In 11 evaluable subjects with proliferative CMML and RAS-pathway mutations, GM-CSF neutralization with LENZ in addition to AZA standard of care, resulted in significant decreases in the proportion of KRAS and CBL mutant leukemic cells, accompanied by clinically significant hematologic improvements and a reduction in splenomegaly. Lenzilumab may have efficacy in preventing outgrowth of RAS-pathway mutations, specifically KRAS and CBL, in the context of CMML and other myeloid malignancies.

### Example 4

Cytokine and Mutation Profiling Reveal Patterns of Complete Remission Rates with Lenzilumab Combination Therapy in Chronic Myelomonocytic Leukemia

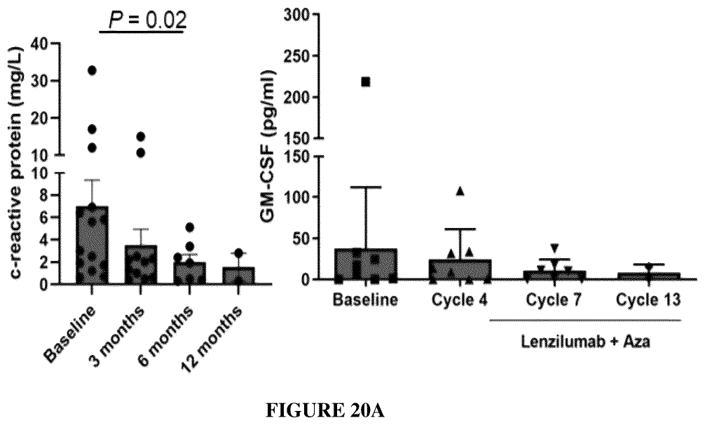

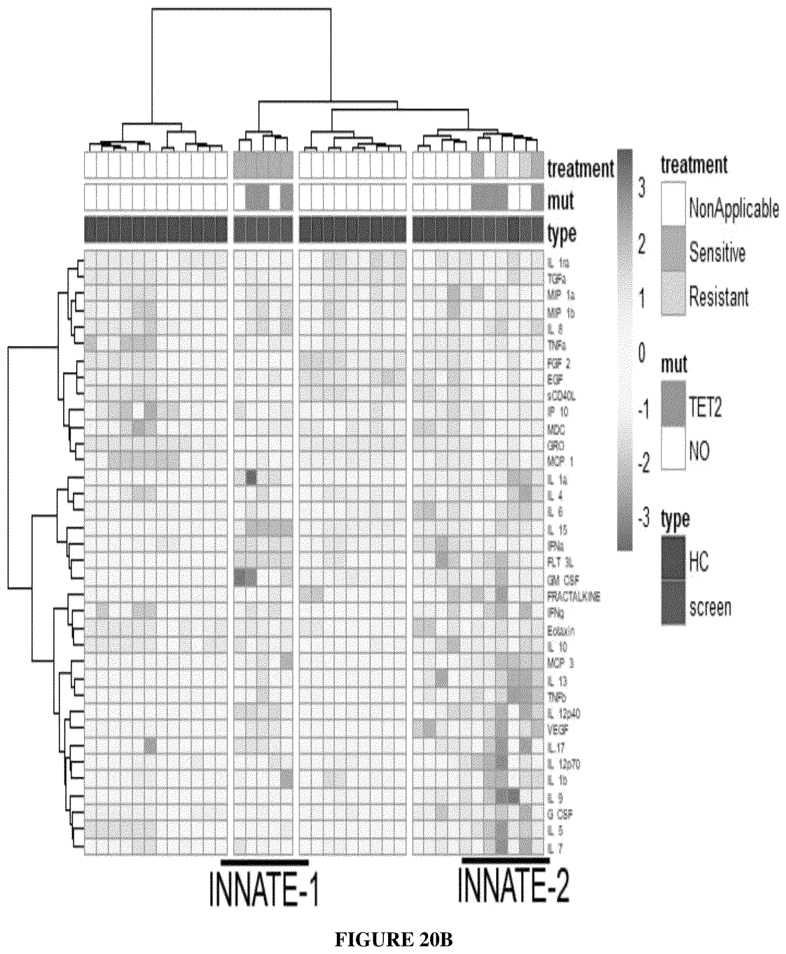

Chronic myelomonocytic leukemia (CMML) is characterized by accumulation of classical CD14+CD16− inflammatory monocytes driven in part by hypersensitivity to granulocyte-macrophage colony-stimulating factor (GM-CSF), a pro-inflammatory cytokine. Standard of care in CMML includes hypomethylating agents such as azacytidine (AZA), with complete response (CR) rates of 16-21% (as described by Xu Y., et al. 2022 supra and Zheng X., et al., 2022 supra) and no reliable biomarkers that predict response. The complete pro-inflammatory profile of CMML is unknown and no treatment addresses the hematologic aberrations of CMML. Lenzilumab (LENZ; Humanigen, Inc., Short Hills, NJ) is a proprietary Humaneered® first-in-class monoclonal antibody with best-in-class off-rate and affinity that neutralizes GM-CSF. The interim analysis of the PREcision Approach to CHronic Myelomonocytic Leukemia (PREACH-M; ACTRN12621000223831) trial showed LENZ/AZA treatment in 11 subjects with RAS-pathway (NRAS/KRAS/CBL) mutations resulted in 8 subjects with complete response or optimal marrow response and improvements in hematological parameters. This Example describes the cytokine profiles and systemic C-reactive protein levels of these subjects. (FIG. 20A)

### Methods

PREACH-M is a Phase 2/3 non-randomized, open-label trial in 72 subjects, aged at least 18 years, with newly diagnosed CMML based on the WHO 2016 criteria; and RAS-pathway mutations at a variant allele frequency ≥3%. Subjects received 24 cycles (every 28 days) of AZA (SC; 75 mg/m² for 7 days) and LENZ (IV; 552 mg; d1 & d15 of cycle 1 and d1 only for all subsequent cycles). Subjects without RAS-pathway mutations received the same AZA regimen and sodium ascorbate (IV; 30 g for 7 days (15 g for 1st dose only; 30 g thereafter if no evidence of tumor lysis syndrome); PO; 1.1 g on all other days). Cytokine profiling from bone marrow plasma was performed after 4, 7, 12 and 24 months using Milliplex Human Cytokine/Chemokine Magnetic bead panel and compared with 24 age-matched healthy subjects. Unsupervised hierarchical clustering with Ward's method sought distinct CMML patterns based on cytokine expression. C-reactive protein (CRP) was determined from blood samples using a routine assay. Variant allele frequencies were determined from bone marrow mononuclear cells using a 41-myeloid panel using Illumina Hi-Seq with a depth of 1000×.

### Results

As of July 2023, 15 subjects were enrolled in the LENZ/AZA arm (8 females, 7 males with mean age 69; mean white cell count, 21×10⁹/L; mean Hb, 121 g/L, mean platelet count; 74×10⁹/L, mean blast count, 10.1%). CRP decreased from a median of 5.6 at baseline to 2.1 mg/L after 6 months of LENZ/AZA (P=0.02). (FIG. 20A)

GM-CSF and other pro-inflammatory cytokines were increased in CMML bone marrow plasma compared with healthy subjects. Hierarchical clustering of all subjects revealed 2 patterns of CMML, distinct from healthy subjects. Cluster “INNATE-1” was comprised of medium increases in inflammatory cytokines associated with innate immune response and M1 macrophage activation (IFN-g, IL-1b, TNFa, IL-12p70, IL-12p40, IL-17, Fractalkine, MCP-3). Cluster “INNATE-2” was associated with extreme increases of these cytokines. The mean proinflammatory innate immune score (sum of z-scores) was 20-fold greater in INNATE-2 compared with INNATE-1 (P=0.055). Somatic mutations SRSF2, WT1, PHF6 were enriched in INNATE-2. All patients (5/5) in INNATE-1 showed 100% response (CR or optimal marrow response resulting in <5% blasts) to AZA/LENZ. (FIG. 20B) Two subjects (2/6) in INNATE-2 demonstrated partial responses or hematological improvement thus far.

### Conclusion

CMML is a disorder of profound innate immune activation, driven by GM-CSF and other pro-inflammatory cytokines. Early treatment with LENZ/AZA, a precision immunotherapeutic approach, leads to a) efficacy in INNATE-1 (FIG. 20B) that exceeds historical CR rates for hypomethylating agents (as described by Xu Y., et al. 2022 supra and Zheng X., et al., 2022 supra); and b) evolving efficacy in INNATE-2, in which pro-inflammatory activity is more robust.

### Example 5

Durable Responses Observed in Chronic Myelomonocytic Leukemia Treated with Lenzilumab and Azacitidine

Chronic myelomonocytic leukemia (CMML) is a rare cancer orchestrated by granulocyte-macrophage colony-stimulating factor (GM-CSF), a pro-inflammatory cytokine that drives leukemic monocyte proliferation. Standard of care (SOC) for CMML treatment includes azacitidine (AZA), with a complete response (CR) rate of 16-21%. The PREcision Approach to Chronic Myelomonocytic Leukemia (PREACH-M;ACTRN12621000223831) trial investigates novel CMML therapies directed by molecular profiling. Lenzilumab (LENZ; Taran Therapeutics, Toms River, NJ) a proprietary Humaneered® first-in-class monoclonal antibody with best-in-class off-rate and affinity that neutralizes GM-CSF. PREACH-M interim results show that LENZ/AZA improves hematologic parameters, decreases spleen size, and dampens pro-inflammatory responses in CMML with RAS-pathway mutations. This report details the objective clinical responses from an interim analysis of the first 20 subjects who completed at least three months LENZ/AZA treatment.

### Methods

PREACH-M is a Phase 2/3 non-randomized, uncontrolled, open-label trial in 54 adults aged at least 18 years, newly diagnosed with WHO 2016 criteria for CMML stratified according to mutation status. Subjects exhibiting RAS-pathway mutations (NRAS, KRAS, CBL) receive 24 cycles (every 28 days) of AZA (SC; 75 mg/m2 for 7 days) and LENZ (IV; 552 mg; d1 & d15 of cycle 1 and d1 only for all subsequent cycles); while those with only TET2 mutations receive the same AZA regimen and sodium ascorbate (IV; 30 g for 7 days [15 g for 1st dose only, 30 g thereafter if no evidence of tumor lysis syndrome]; PO; 1.1 g on all other days). Subjects who complete 24 cycles of treatment are followed every 6 months for an additional 24 months. The primary endpoint is the frequency of complete response (CR) or partial response (PR) during the first 12 cycles according to Savona Criteria. Secondary endpoints include responses according to modified 2006 International Working Group criteria, 2 year overall survival, and symptom improvement. Interim analyses are planned when at least 33% (n=18) and 66% (n=36) patients have completed 12 months of treatment.

### Results

As of Aug. 1, 2024, 27 subjects were enrolled overall (18 receiving>12 months of treatment) and 20 subjects were enrolled in the LENZ/AZA arm (9 females, 11 males with mean age 69; mean white cell count 37.8×10⁹/L, mean Hb 108 g/L; mean platelet count, 72×10⁹/L, mean blast count, 9%). Mutations included CBL (65% of subjects), NRAS (20%), KRAS (50%), ASXLI (55%) and TET2 (70%). Subjects exhibited CPSS-MOL scores, of intermediate risk 1 (n=2), intermediate risk 2-3 (n=12), and high risk 4-6 (n=6). Overall, subjects had completed a median number of 13.5 cycles of LENZ/AZA at the time of reporting. 14 (70%) of subjects had not progressed, 2 went to allogeneic transplant and 4 (20%) had progressive disease. 85% of patients attained a complete remission (CR) or marrow complete remission (mCR) within the first 12 months on study, according to IWG 2006 criteria. According to Savona criteria, 85% of patients achieved a complete response or an optimal marrow response within the first 12 months on treatment. Importantly, 64% of patient achieving CR or marrow CR had detectable CBL mutations at baseline. Of the 10 subjects with dominant CBL mutations (VAF>10%), 90% achieved a CR or mCR in first 12 months with durable suppression of CBL clones. Of the 11 subjects with major clone KRAS/NRAS mutations (VAF>10%), 81% achieved a CR or mCR. Significantly, of all subjects who achieve CR or mCR at 3 months, 83% were still on study without progression at 12 months. Safety and tolerability for LENZ was excellent with no patients experiencing infusion related reactions. A total of nineteen grade 3 or 4 serious adverse events were reported, including anemia and febrile neutropenia, none of which were considered causally related to LENZ and six possibly related to azacitidine.

### Conclusion

Interim analysis of the PREACH-M trial shows promising results with LENZ/AZA resulting in durable complete responses beyond 12 months with 85% of subjects achieving a complete remission or marrow complete remission without significant LENZ related toxicity. Significantly, 90% of patients with major clone CBL mutations achieved complete remission or marrow complete remission.

### Example 6

### Suppression of KRAS and CBL Mutations and Hematological Improvement by Lenzilumab and Azacitidine Treatment in Proliferative Chronic Myelomonocytic Leukemia

Somatic mutations in RAS-pathway genes (NRAS, KRAS, CBL) are common in chronic myelomonocytic leukemia (CMML), with historically resistant to most therapies, and high propensity to develop into acute myeloid leukemia, which makes targeted therapies against RAS-pathway a major unmet need in oncology.

In pre-clinical models, Lenzilumab (LENZ; Taran Therapeutics, Toms River, NJ), a proprietary Humaneered® first-in-class monoclonal antibody that neutralizes GMCSF, resulted in a reduction of colony numbers and viability of CMML cells, with the greatest sensitivity in cells possessing RAS-pathway mutations.

The PREcision Approach to Chronic Myelomonocytic Leukemia (PREACH-M) trial assesses the efficacy of LENZ and azacytidine (AZA), in CMML subjects with RAS-pathway mutations and high dose sodium ascorbate (ASC) and AZA in CMML subjects without RAS-pathway mutations.

The aim of this study was to assess the variant allele frequencies (VAFs) for RAS-pathway mutations and hematologic changes associated with LENZ/AZA treatment in CMML.

### Methods

PREACH-M is a Phase 2/3 nonrandomized, open-label trial in 72 subjects, aged at least 18 years, with newly diagnosed CMML based on WHO 2016 criteria (n=19).

VAFs was determined from bone marrow mononuclear cells using a 41-myeloid panel using Illumina Hi-Seq with a depth of 1000× performed on bone marrow aspirates obtained at baseline and Day 1 of treatment cycles 4, 7, and 12.

Subjects received 24 cycles (every 28 days) treatments according to their mutation profiling:

Confirmed NRAS/KRAS or CBL Mutation at VAF ≥3% (n=15) Lenzilumab (IV; 552 mg; day 1 & day 15 of cycle 1 and day 1 only for all subsequent cycles) and Azacitidine (SC; 75 mg/m² for 7 days).
Confirmed TET2 mutation and without RAS-pathway mutation (n=4) Ascorbic acid (ASC) (IV; 30 g for 7 days (15 g for 1st dose only; 30 g thereafter if no evidence of tumor lysis syndrome); PO; 1.1 g on all other days) and Azacitidine (SC; 75 mg/m2 for 7 days).

If no mutation in either category, the screen was a fail. Data are updated since 31st of October 2023.

### Results

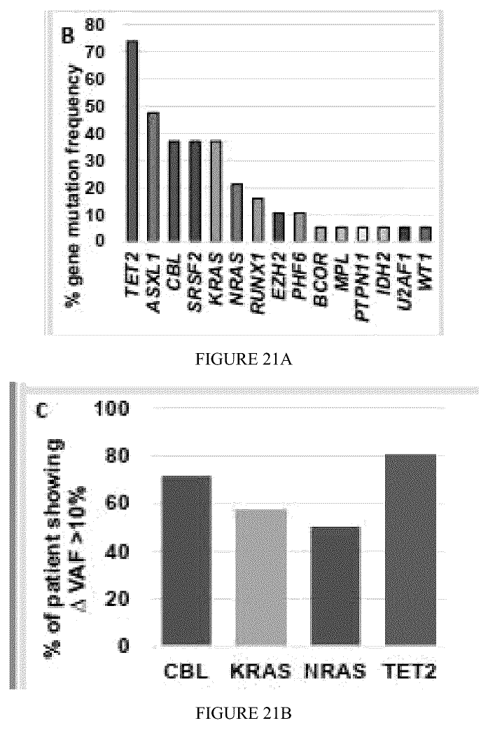

FIG. 19 shows a CIRCOS plot of co-occurring somatic mutations detected at VAF>3% in all patients enrolled in PREACH-M at screening. FIG. 21A shows a baseline gene mutation frequency in PREACHM. FIG. 21B shows the percentage of subjects showing VAF reduction >10%.

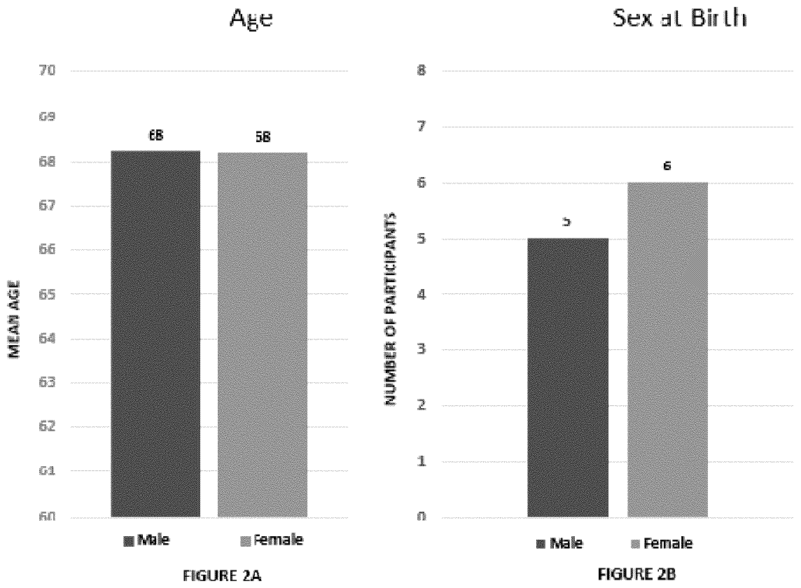

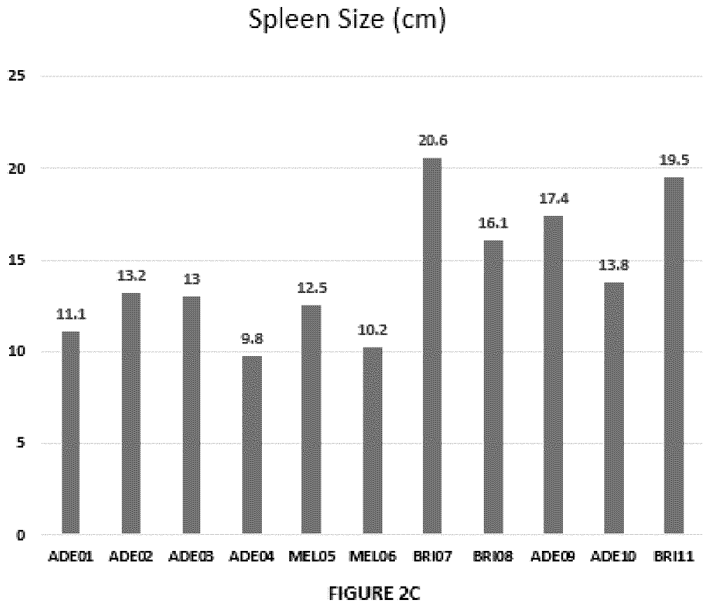

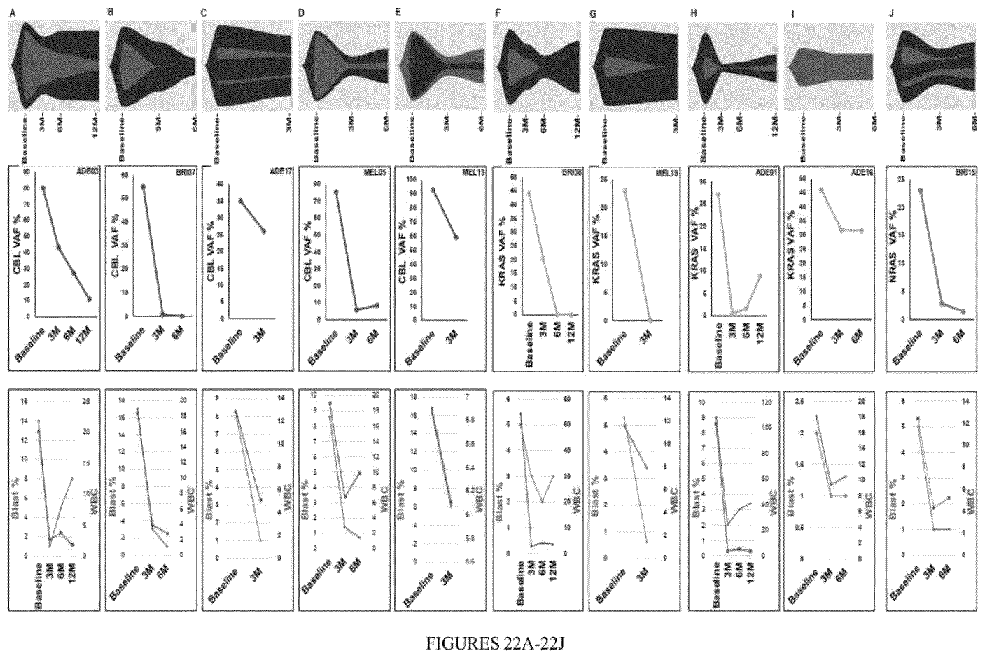

FIGS. 22A-2J show Fish plots showing decrease in VAF (in red) compared to parent clone (in blue). Line graphs showing decrease of RAS-pathway genes VAF % in 9 subjects after LENZ/AZA treatment in second row. Line graph tracking the percentage of blasts and WBC over time in third row.

### Conclusions

In 15 evaluable subjects with proliferative CMML and RAS pathway mutations, GM-CSF neutralization with LENZ in addition to AZA standard of care, resulted in significant decreases in the proportion of KRAS and CBL mutant leukemic cells, accompanied by clinically significant hematologic improvements and a reduction in splenomegaly. Lenzilumab may have efficacy in preventing outgrowth of RAS-pathway mutations, specifically KRAS and CBL, in the context of CMML and other myeloid malignancies. Updated data since Oct. 31, 2023 is shown in FIGS. 22A-22J showing multiple patients with decreases in RAS pathway clones.

### Example 7

### Lenzilumab in Addition to Azacitidine Improves Complete Response Rates in Chronic Myelomonocytic Leukemia

Chronic myelomonocytic leukemia (CMML) is a rare cancer orchestrated by granulocyte-macrophage colony-stimulating factor (GM-CSF), a proinflammatory cytokine that drives leukemic monocyte proliferation. Standard of care (SOC) for CMML treatment includes azacitidine (AZA), with a complete response (CR) rate of 16-21%. The PREcision Approach to Chronic Myelomonocytic Leukemia (PREACH-M; ACTRN12621000223831) trial investigates novel CMML therapies directed by molecular profiling. Lenzilumab (LENZ; Taran Therapeutics, Toms River, NJ) a proprietary Humaneered® first-in-class monoclonal antibody with best-in-class off-rate and affinity that neutralizes GM-CSF.

This study details the objective clinical responses from an interim analysis of the first 15 subjects who completed at least three months LENZ/AZA treatment, updated as of database lock Oct. 31 2023.

### Methods

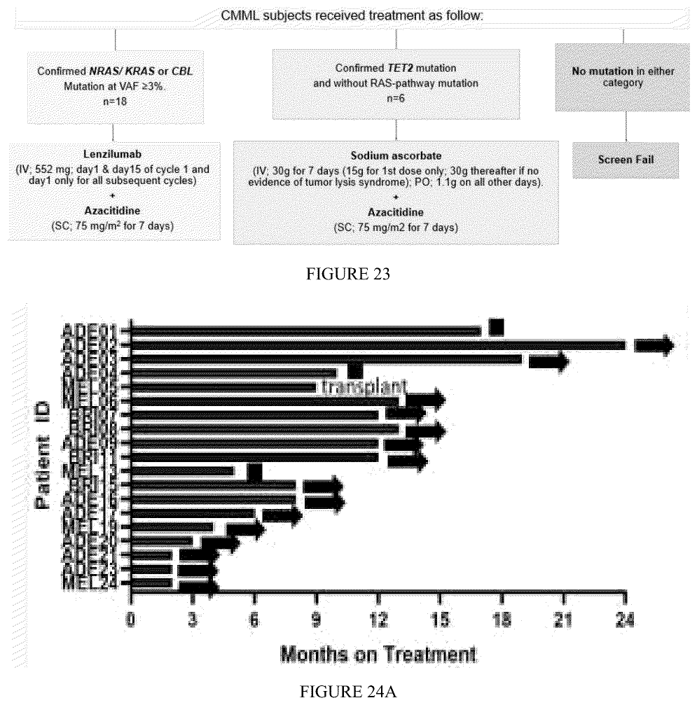

FIG. 23 shows the treatment the CMML subjects received. Confirmed NRAS/KRAS or CBL Mutation at VAF≥3% (n=15) Lenzilumab (IV; 552 mg; day 1 & day 15 of cycle 1 and day 1 only for all subsequent cycles) and Azacitidine (SC; 75 mg/m2 for 7 days). Confirmed TET2 mutation and without RAS-pathway mutation (n=4) Ascorbic acid (ASC) (IV; 30 g for 7 days (15 g for 1st dose only; 30 g thereafter if no evidence of tumor lysis syndrome); PO; 1.1 g on all other days) and Azacitidine (SC; 75 mg/m2 for 7 days). If no mutation in either category, the screen was a fail.

### Results

As of Oct. 31, 2023, 18 patients were enrolled in the LENZ+AZA arm with median 8 months on study. 14 patients remain on study, 3 having progressed and 1 undergoing allogeneic stem cell transplant. 6 patients were enrolled on the ASCORBATE+AZA arm, none having progressed.

According to Savona criteria, of 14 evaluable patients with response data and baseline blasts >5%, 78% (11 patients) achieved either a complete response or an optimal marrow response within the first 12 months. 5 patients (36%) achieved a complete response, 6 patients (42%) achieved an optimal marrow response.

According to 2006 IWG criteria, of 14 evaluable patients with response data and baseline blast count >5% that received LENZ+AZA, 78% (11 patients) achieved CR or marrow CR at some point within the first 12 months. 7 out of 14 (50%) achieved CR with complete hematological recovery within 12 months and 4 out of 14 achieved a marrow CR. Table 1A shows the baseline characteristics of subjects included in PREACH-M.

TABLE 1A
Baseline characteristics of subjects included in PREACH-M
Lenz cohort n = 18

| Age | 72 | | |
|---|---|---|---|
| Sex | M/F | 11 (61%)/7 (39%) | |
| CMML 0, 1, 2 | 0 (0%), 3 (17%), | | |
| | 15 (83%) | | |
| Median Hemoglobin | g/L | 107 | (82-143) |
| Median Platelet count | ×10\hat{ }9/L | 74 | (18-192) |
| Median Marrow Blasts | (%) | 8.3 | (2-17) |
| Median WCC | ×10\hat{ }9/L | 19.4 | (6.9-103.3) |
| Median Monocytes | ×10\hat{ }9/L | 5.6 | (0.7-47.5) |
| Median Spleen size | cm | 14.7 | (9.8-20.6) |
| Red cell transfusion | 1 (5%) | | |
| dependent | | | |

Ascorbate cohort n = 6

| Age | 75 | | |
|---|---|---|---|
| Sex | M/F | 2 (33%), 4 (67%) | |
| CMML 0, 1, 2 | 1 (17%), 3 (50%), | | |
| | 2 (33%) | | |
| Median Hemoglobin | g/L | 110 | (79-124) |
| Median Platelet count | ×10\hat{ }9/L | 83.5 | (7-219) |
| Median Marrow Blasts | (%) | 4.5 | (1-10.3) |
| Median WCC | ×10\hat{ }9/L | 7.4 | (4.9-36.8) |
| Median Monocytes | ×10\hat{ }9/L | 1.98 | (1.02-7.97) |
| Median Spleen size | cm | 11.7 | (10.9-13.8) |
| Red cell transfusion | 2 (25%) | | |
| dependent | | | |

FIGS. 24A-24B show Lenzilumab in addition to Azacitidine improves complete response rates in CMML. FIG. 24A shows a Swimmer plot showing ongoing treatment of patients with RAS-pathway mutations on LENZ/AZA arm of the study. Black arrow indicates patient has not progressed. FIG. 24B shows column graphs showing decreased bone marrow blast % with LENZ/AZA treatment assessed after 3, 6, 12 and 24 months of combination treatment. P values reflect unpaired students t-test for groups as shown.

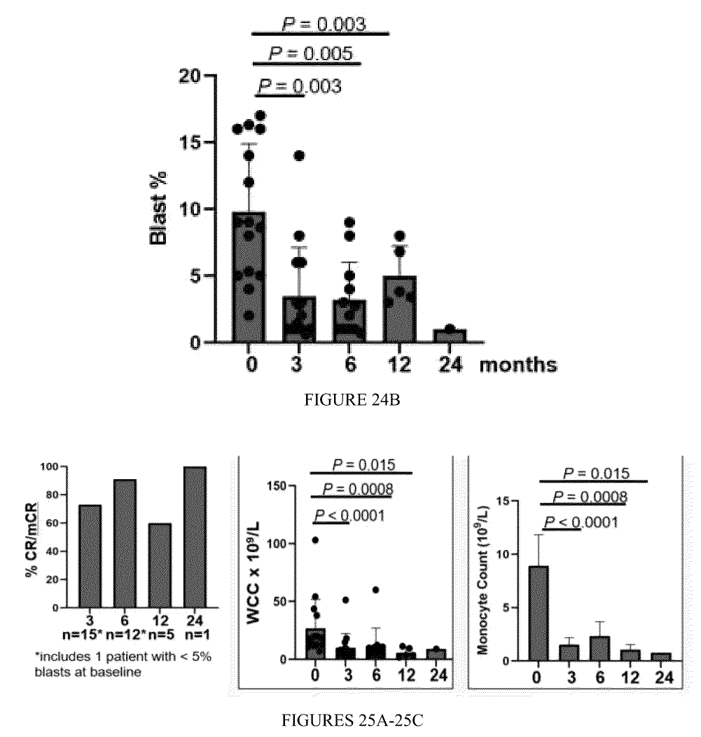

FIGS. 25A-25C show durable responses with ongoing CR or marrow CR (IWG criteria) for patients in LENZ-AZA cohort (FIG. 25A) and durable decrease in white cell count (FIG. 25B) and monocyte count (FIG. 25C).

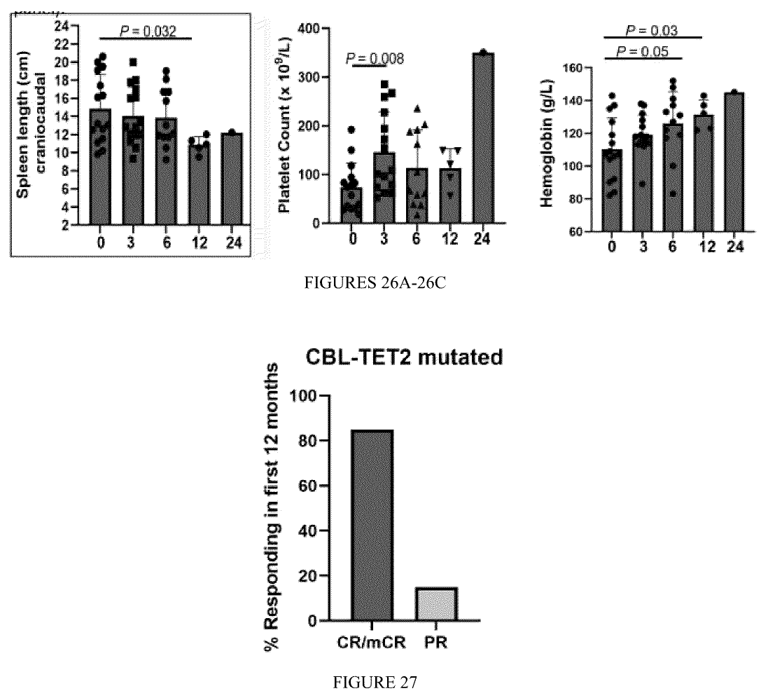

FIGS. 26A-26C show durable improvements in spleen length (FIG. 26A), platelet count (FIG. 26B), and hemoglobin (FIG. 25C). P values indicate unpaired Whitney-U non-parametric test.

Table 1B shows the patient responses to treatment according to 2006 IWG criteria.

TABLE 1B
Responses to treatment according to 2006 IWG criteria

| Responses (IWG 2006 Criteria) | | | | | |
|---|---|---|---|---|---|
| Updated Oct 31 | | Responses (Savona Criteria) | | Responses (Wattel et al) | |
| CR | 7 (50%) | Complete Response | 5 (36%) | Clinical Remission | 6 (40%) |
| PR | 2 (14%) | Optimal Marrow Response | 6 (43%) | Minor Response | 5 (33%) |
| Marrow CR | 4 (29%) | Partial Marrow Response | 2 (14%) | Good Response | 3 (20%) |
| Stable disease | 1 (7%) | Progressive disease | 1 (7%) | Stable disease | 1 (6%) |
| Progressive disease | 1 (7%) | CR + optimal MR | 11 (78%) | CR + GR | 9 (60%) |
| CR + marrow CR | 11 (78%) | | | | |

### Conclusions

Updated interim analysis of the PREACH-M trial demonstrated that GM-CSF neutralization with LENZ/AZA, for the treatment of CMML with RAS-pathway mutations resulted in 78% achieving CR or marrow CR (IWG criteria), early in treatment, durability up to 24 months with evidence of ongoing response (CR or marrow CR) in some patients. These data suggest CMML is driven by a non-redundant cytokine that responds to immunotherapy.

### Example 8

Cytokine and Mutation Profiling Reveal Patterns of Complete Remission Rates with Lenzilumab Combination Therapy in Chronic Myelomonocytic Leukemia

Chronic myelomonocytic leukemia (CMML) is characterized by accumulation of classical CD14+CD16− inflammatory monocytes driven in part by hypersensitivity to granulocyte-macrophage colony-stimulating factor (GMCSF), a pro-inflammatory cytokine. Standard of care in CMML includes hypomethylating agents such as azacytidine (AZA), with complete response (CR) rates of 16-21% and no reliable biomarkers that predict response. The PREcision Approach to Chronic Myelomonocytic Leukemia (PREACH-M; ACTRN12621000223831) trial investigates novel CMML therapies directed by molecular profiling. Lenzilumab (LENZ; Taran Therapeutics, Toms River, NJ) is a proprietary Humaneered first-in-class monoclonal antibody with best-in-class off rate and affinity that neutralizes GM-CSF. The complete pro-inflammatory profile of CMML is unknown and no treatment addresses the hematologic aberrations of CMML.

The aim of this study was to investigate somatic mutations profile, measure cytokine levels and systemic C-reactive protein levels of 13 PREACH-M subjects at different time points (Baseline, Cycle 4, 7 and 13).

### Methods

Newly diagnosed CMML patients (n=13), of which 11 were confirmed as having RAS mutations (NRAS, KRAS or CBL with or without TET2 mutations) were treated with Lenzilumab (IV; 552 mg; day 1 & day 15 of cycle 1 and day 1 only for all subsequent cycles) for 24 cycles (28 days, each cycle) and Azacitidine (SC; 75 mg/m² for 7 days).

Patients confirmed as having TET2 mutation only (n=2) were treated with Azacitidine (SC; 75 mg/m² for 7 days) and Ascorbic acid (sodium ascorbate, ASC) (IV; 30 g for 7 days (15 g for 1st dose only; 30 g thereafter if no evidence of tumor lysis syndrome); PO; 1.1 g on all other days).

Cytokine levels and systemic C-reactive protein levels were assessed at baseline, and Cycle 4, 7 and 13. Bone marrow plasma was used for cytokines array, blood was tested for C-reactive protein levels, and bone marrow mononuclear cells were used for mutational studies.

### Results

FIG. 19 shows the prevalence of RAS and TET2 mutations at baseline in CMML patients: Circos plot of co-occurring somatic mutations detected at variant alleles with greater than 3% frequency, in all patients enrolled in the study to date. RAS pathway mutations CBL and KRAS were associated with other non-RAS mutations such as TET2 while the single NRAS mutation was singularly associated with PHF6 mutation. FIG. 20B shows that the baseline cytokine profile can predict response to LENZ/AZA treatment: Heatmap showing hierarchical clustering of cytokines levels measured at baseline compared to age-matched healthy controls (HC). Two discrete clusters of CMML are apparent, INNATE-1 and INNATE-2. Patients with early response shown in orange blocks (sensitive).

FIG. 27 shows that patients with CBL-TET2 mutations are better responders to LENZ/AZA treatment. Within first 12 months of LENZ/AZA treatments, CMML patients with CBL-TET2 mutation showed excellent responses with a decrease in bone marrow blasts. CR-complete remission; mCR-marrow complete remission; PR-partial remission.

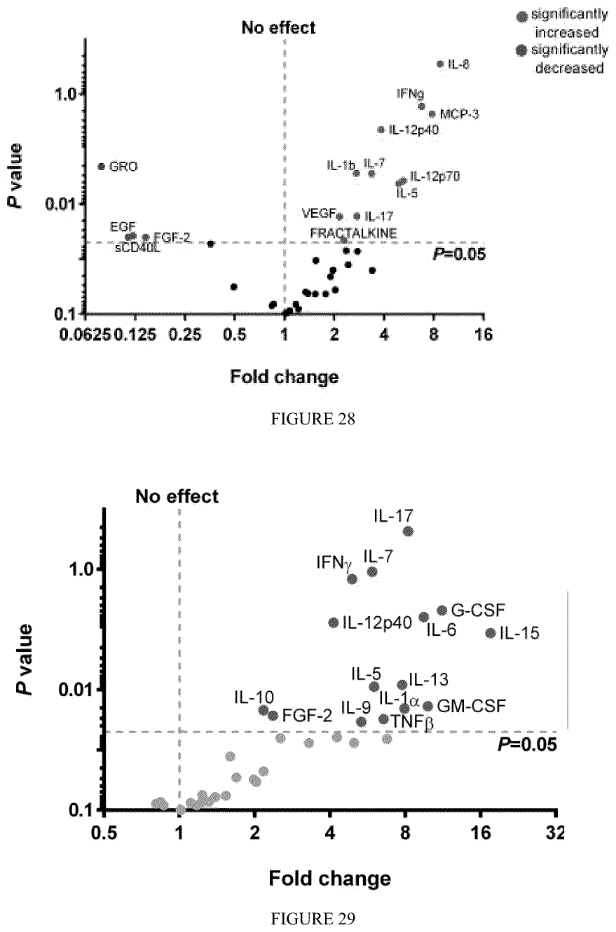

FIG. 28 shows a pro-inflammatory environment at baseline in CML patients: a Volcano plot illustrating cytokines levels in bone marrow plasma of CMML patients at baseline compared to age-matched healthy controls.

FIG. 29 shows that a cytokines profile at baseline can predict response to LENZ/AZA treatment: a Volcano plot representation of higher inflammatory cytokines levels in bone marrow plasma of patients resistant to LENZ/AZA treatment compared to patients responding to treatment.

FIG. 20B shows a heatmap showing hierarchical clustering of cytokines levels measured at baseline compared to age-matched healthy controls (HC). Two discrete clusters of CMML are apparent, INNATE-1 and INNATE-2. Patients with early response shown in orange blocks (sensitive).

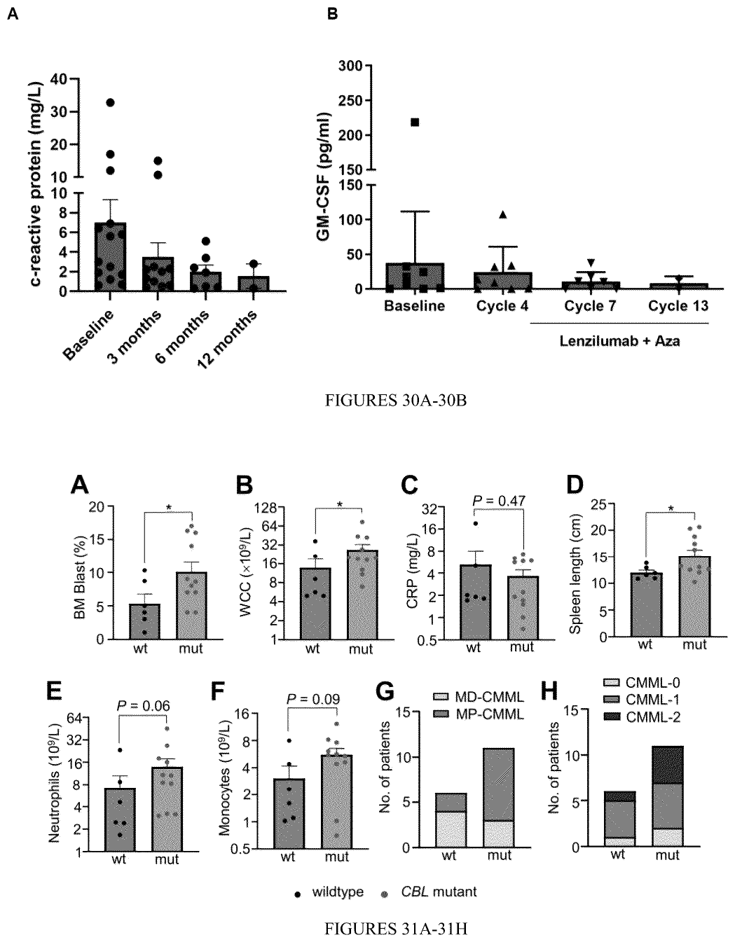

FIGS. 30A-30B shows inflammatory parameters improvement in CMML patients after treatment with LENZ/AZA. FIG. 30A depicts column graphs showing a decrease in C-reactive protein from baseline and after 3, 6 and 12 months after LENZ/AZA treatment. FIG. 30B depicts column graphs showing bone marrow GM-CSF after 4, 7 and 13 cycles of LENZ/AZA treatment.

### Conclusions

CMML is a disorder of profound innate immune activation, driven by GM-CSF and other pro-inflammatory cytokines. Early treatment with LENZ/AZA, a precision immunotherapeutic approach, leads to (a) efficacy in INNATE-1 that exceeds historical CR rates for hypomethylating agents (Zheng X, et al., Efficacy and Safety of Hypomethylating Agents in Chronic Myelomonocytic Leukemia: A Single-Arm Meta-analysis. Glob Med Genet 2022;9(2):141-151.); and (b) evolving efficacy in INNATE-2, in which pro-inflammatory activity is more robust.

### Example 9

CBL Mutations in Chronic Myelomonocytic Leukemia Often Occur in the RING Domain with Multiple Subclones Per Patient: Implications for Targeting

Monocytes give rise to tissue macrophages that can perform a myriad of biological functions ranging from innate immune activation to phagocytosis and wound healing. Chronic myelomonocytic leukemia (CMML) is a rare blood cancer of older adults (3 in every 1,000,000 persons) characterized by an increase in clonal CD14⁺ CD16⁻ classical monocytes and their precursors in the blood and bone marrow. Because of its subtle presentation, the disease is often diagnosed late, but is likely to rise in prevalence due to routine uptake of next-generation sequencing (NGS) in some hospitals, new monocytosis criteria by the World Health Organization and increasing recognition by physicians, especially in persons previously treated with cytotoxic therapy (therapy-related CMML). Many patients present with autoinflammatory features such as vasculitis, polychondritis, Sweet syndrome and pleural/pericardial effusions but the mechanism and intersection of clonal monocytes and innate vs. adaptive immunity is not understood.

The molecular pathogenesis of CMML is only beginning to be characterized. Though 70% of CMML patients present without any cytogenetic abnormalities they harbor somatic mutations in genes that influence epigenetic regulation (TET2, DNMT3A, ASXL1, EZH2, IDH1, IDH2), mRNA splicing (SRSF2, U2AF1), genome stability (SETBP1, TP53), transcription regulation (RUNX1, CEBPA, NPM1) and cell signaling pathways (KRAS, NRAS, CBL, PTPN11, JAK2, MPL). Interestingly, CMML shares some biological and morphological features with a clonal disease occurring in young children, juvenile myelomonocytic leukemia (JMML). Around 90% of cases of JMML are associated with mutations in the RAS signaling pathway (PTPN11, NRAS, KRAS, NF1 and CBL). Notably, while the overall molecular patterns of CMML and JMML are distinct, mutations in CBL are found with equal frequency, at approximately 15%, in both diseases. Both CMML and JMML display hypersensitivity to the pro-inflammatory cytokine granulocyte-macrophage colony-stimulating factor (GM-CSF) which promote the differentiation of classical pro-inflammatory monocytes. Emerging reports suggest that CBL mutations are associated with inferior survival in both CMML and JMML.

The CBL gene is located on 11q23.3 and encodes an E3 ubiquitin ligase (c-Cbl or Cbl) that acts as both a positive and negative regulator in the signal transduction of activated receptor tyrosine kinases (RTKs) and cytokine receptors. Cbl relays signals downstream of activated RTKs by functioning as an adaptor, and at the same time, attenuates signaling by promoting the ubiquitination of RTKs through its E3 ligase activity, marking them for degradation by the proteasome or via endocytosis. Cbl recognizes phosphorylated tyrosines on active RTKs through its Src homology 2 domain within the tyrosine kinase binding domain (TKBD), and binds E2 ubiquitin-conjugating enzymes though its conserved RING domain. The TKBD and RING domain are connected by a 35 amino acid sequence, referred to as the linker helix region (LHR). A proline-rich region, serine-rich region, several C-terminal principal phosphorylation sites (Y674, Y700, Y731, Y774) and a ubiquitin-association domain (UBA) complete the structure of Cbl. Several studies on the role of the LHR and RING domain have shown that mutation of these domains could lead to loss of activity and/or gain of oncogenicity.

To date, there has not been significant mutation-specific therapy developed for CMML, unlike chronic myeloid leukemia, and standard-of-care with hypomethylating agents azacitidine or decitabine is not curative. While allogeneic hematopoietic stem cell transplantation may be potentially curative, stem cell transplantation is not a viable option for most older CMML patients. Currently survival is estimated at a median of 31 months, with even shorter life expectancy for patients with CMML-2 (more than 10% bone marrow blasts/promonocytes). Transformation to acute myeloid leukemia (AML), an aggressive cancer with poor long-term survival, occurs in up to 20% of CMML patients within 5 years.

In this study, the clinical presentation of CMML patients with CBL mutations enrolled in a prospective clinical trial were analyzed. Strikingly, 7 out of 11 (64%) patients were found to have more than one CBL clone, implying a complex clonal architecture. Clinically, CBL mutations were associated with a more proliferative phenotype evidenced by increased bone marrow (BM) blasts, leukocytosis and splenomegaly, similar to other RAS pathway mutations such as KRAS, NRAS and PTPN11. It also was found that CMML CBL mutations often co-occurred with TET2 mutations and were enriched in the RING domain compared to the LHR (P<0.0001). Furthermore, an increased percentage of CD116 and CD131 positive CMML CD34⁺ stem and progenitors was noted compared to healthy controls. In summary, the data herein suggest that CBL mutants are associated with distinct clinical and molecular features in CMML. These differences highlight the potential for targeted therapy with CD116-directed immunotherapy or targeted protein degradation mechanisms.

### Materials and Methods

### Patients

Between 1 Oct. 2021 and 30 Sep. 2023, 24 patients with CMML, diagnosed according to the 2016 WHO Classification of Myeloid Neoplasms, met the eligibility criteria for the trial (untreated CMML with high white cell count, cytopenia or constitutional symptoms). Detection of TET2, KRAS, NRAS or CBL mutation at a variant allele frequency (VAF) percentage of ≥3% was a key inclusion criteria. Of these patients, 13 were male and 11 were female. The median age was 73 years (range 56-86 years). Written informed consent for genetic analysis and use of laboratory results for scientific research were obtained during trial enrolment. The trial was approved in multiple centers across Australia including the Royal Adelaide Hospital, Royal Brisbane and Women's Hospital and Austin Health. The trial was conducted with approval from the Royal Adelaide Hospital human research ethics committee (2021/HRE00017) and registered on the Australian and New Zealand Clinical Trials Registry (ANZCTR) (Registration number ACTRN12621000223831, Acronym PREACH-M).

### Clinical Presentation

All clinical and laboratory data were acquired at screening, prior to commencement of the therapy protocol outlined in the trial. Complete blood examination and bone marrow (BM) analyses were performed for each patient. The spleen craniocaudal length was determined by ultrasonography.

### Mutation Screening

Targeted enrichment of selected coding exons and flanking intronic regions of 46 genes was performed using a custom-designed hematological neoplasms capture panel (Integrated DNA Technologies; HaemV1) and analyzed by next-generation sequencing (NGS) (Illumina NextSeq sequencing system). Variant calling was performed using Vardict and Mutect2 where variants with VAF<5% were reported where clinically significant. These assays were performed by accredited pathology laboratories across Australia.

Cord Blood and Peripheral Blood Mononuclear Cells from Healthy Donors

Umbilical cord blood was collected with written consent from scheduled caesarean section deliveries at the Women's Health Unit, Lyell-McEwin Hospital (Adelaide, South Australia) with human ethics committee approval (HREC/20/WCHN/65). Peripheral blood buffy coat samples were retrieved from Australian Red Cross Lifeblood (Adelaide, South Australia) and studies were approved by the Central Adelaide Local Health Network Human Ethics Committee (CALHN HREC#HREC/15/RAH/448) and conducted in accordance with the Declaration of Helsinki. Samples were processed by density gradient centrifugation using Lymphoprep™ (Stemcell Technologies, USA) to isolate mononuclear cells (MNCs).

### Flow Cytometry

Peripheral blood mononuclear cells (PB-MNCs) collected at screening from patients in the PREACH-M trial were immunophenotyped by spectral flow cytometry (Cytek® Aurora, USA). Control samples include cord blood mononuclear cells (CB-MNCs) and PB-MNCs from healthy donors. All MNCs were incubated with Human TruStain FcX™ (BioLegend, USA) and True-Stain Monocyte Blocker™ (BioLegend, USA) prior to antibody staining. Antibody panel included mouse anti-human BV421 CD45 (HI30), PE-Cy7 CD14 (M5E2), PE-Cy5 CD16 (3G8), APC CD34 (8G12), PE CD114 (LMM741), PE CD116 (hGMCSFR-M1), PE CD131 (1C1); BV421 CD131 (3D7) and rat anti-human PE CD115 (9-4D2-1E4). Viability was determined using Zombie Aqua™ Fixable Dye (BioLegend, USA). A list of catalogue numbers and clones of antibodies can be found in Table 2A and the spleen craniocaudal length (cm) of CBL mutant CMML vs wildtype is shown in Table 2B.

TABLE 2A
List of Antibodies for Flow Cytometry

| Antibody | Clone | Manufacturer | Cat # |
|---|---|---|---|
| BV421 Mouse | HI30 | BD Horizon | 563879 |
| anti-human CD45 | | | |
| PE-Cy ™7 Mouse | M5E2 | BD Pharmingen ™ | 557742 |
| anti-Human CD14 | | | |
| PE-Cy ™5 Mouse | 3G8 | BD Pharmingen ™ | 561725 |
| anti-Human CD16 | | | |
| APC Mouse | 8G12 | BD | 340441 |
| Anti-Human CD34 | | | |
| PE Mouse | LMM741 | BD Pharmingen ™ | 554538 |
| anti-Human CD114 | | | |
| PE Rat anti- | 9-4D2-1E4 | BD Pharmingen ™ | 565368 |
| Human Hu CD115 | | | |
| PE Mouse | hGMCSFR-M1 | BD Pharmingen ™ | 551373 |
| anti-Human CD116 | | | |
| PE Mouse | YB5.B8 | BD Pharmingen ™ | 555714 |
| anti-Human CD117 | | | |
| PE Mouse | 1C1 | Invitrogen | 12-1319-42 |
| anti-Human CD131 | | | |
| BV21 Mouse | 3D7 | BD OptiBuild ™ | 752992 |
| anti-Human CD131 | | | |

TABLE 2B
Spleen craniocaudal length (cm) of CBL mutant CMML vs wildtype

| | | Spleen | |
|---|---|---|---|
| | | length (cm) | Splenomegaly^{#} |
| CBL mutant | MEL13 | 12.6 | Yes |
| | ADE09 | 17.4 | Yes |
| | ADE17 | 16.3 | Yes |
| | BRI07 | 20.6 | Yes |
| | MEL05 | 12.5 | Yes |
| | ADE02 | 13.2 | Yes |
| | ADE03 | 13.0 | Yes |
| | ADE20 | 18.8 | Yes |
| | ADE21 | 20.3 | Yes |
| | MEL06 | 10.2 | No |
| | ADE23 | 12.0 | Yes |
| | Total with splenomegaly | | 10/11 (91%) |
| CBL wildtype | BRI14 | 10.8 | No |
| | MEL18 | 11.9 | No |
| | ADE10 | 13.8 | Yes |
| | ADE22 | 10.9 | No |
| | BRI12 | 12.9 | Yes |
| | MEL24 | 11.5 | No |

| | |
|---|---|
| Total with splenomegaly | 2/6 (33%) |

^{#}Splenomegaly defined as spleen cradiocaudal length equal or greater than 12.0 cm
Fisher's exact test; P < 0.05 statistically significant. P = 0.0276.

### Hotspot and Protein Structure Analysis

Data from the Catalogue of Somatic Mutations in Cancer (COSMIC) were analyzed (last accessed on 15 Feb. 2024). Analysis of CBL mutations were filtered as follows: Primary site hematopoietic and lymphoid, histology hematopoietic neoplasm; sub-histology CMML or JMML. 120 variants were found for CMML, 46 for JMML. Variants were stratified to include nonsense or missense substitutions, frameshift insertion or deletions, and in-frame insertions or deletions, with the exclusion of synonymous mutations, within the coding sequence. The six most common mutations for CMML and JMML were identified. Protein structure visualization and alignment were performed using Maestro 13.8 (Schrodinger, USA).

### Statistical Analysis

Values between unique samples were presented as mean±standard error of margin (S.E.M.) or as mean±standard deviation (S.D.) between technical replicates. For comparisons between groups, Student's t-test or Mann-Whitney test was applied to analyze measurement (continuous) data and Fisher's exact test for enumeration (categorical) data. All statistical analyses were performed using GraphPad Prism 10. P-values for Student's t-tests were two-tailed, Mann-Whitney tests were one-tailed, and Fisher's exact tests were two-tailed. P<0.05 was considered statistically significant.

### Results

CBL Mutations are Associated with Increased Marrow Blasts, Leukocytosis and Splenomegaly, Consistent with RAS Pathway Activation

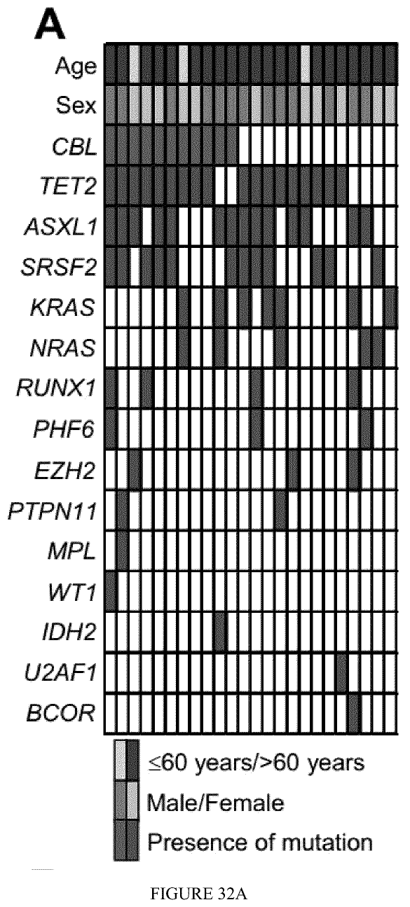

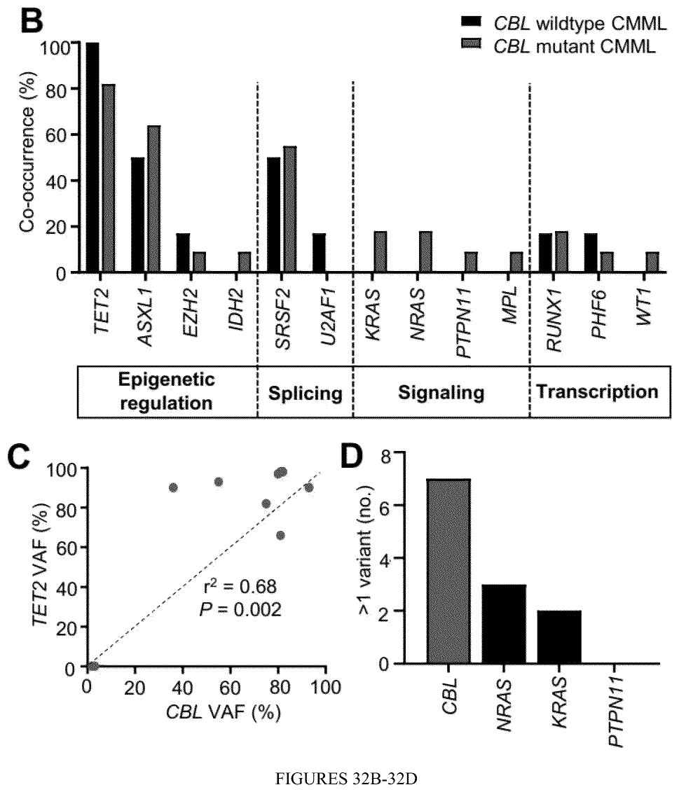

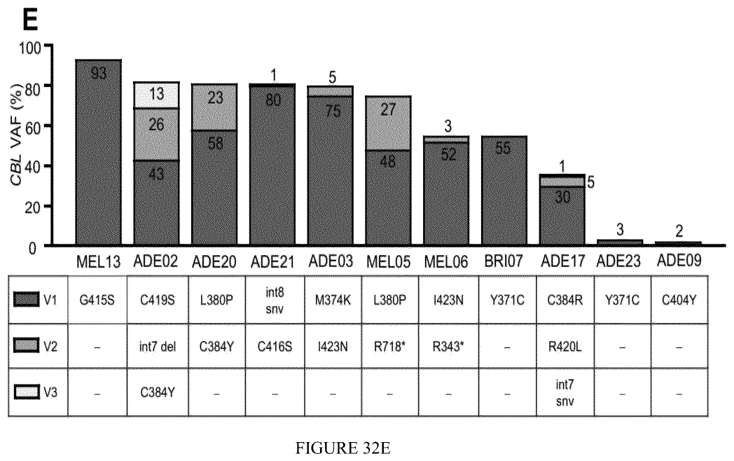

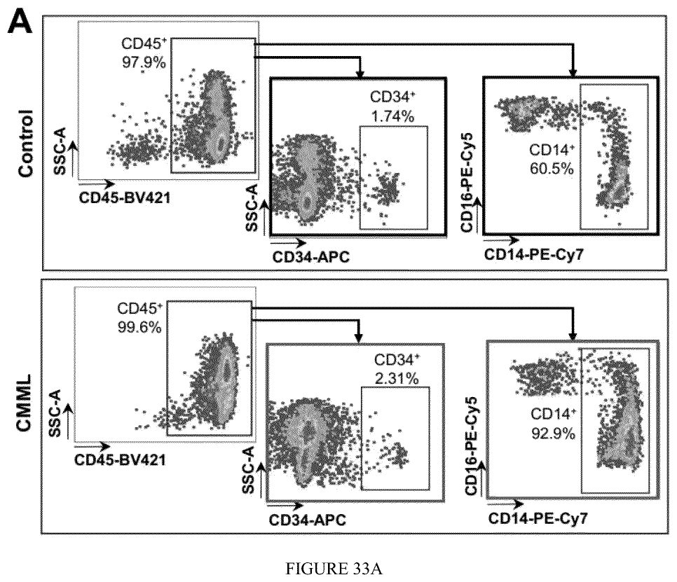

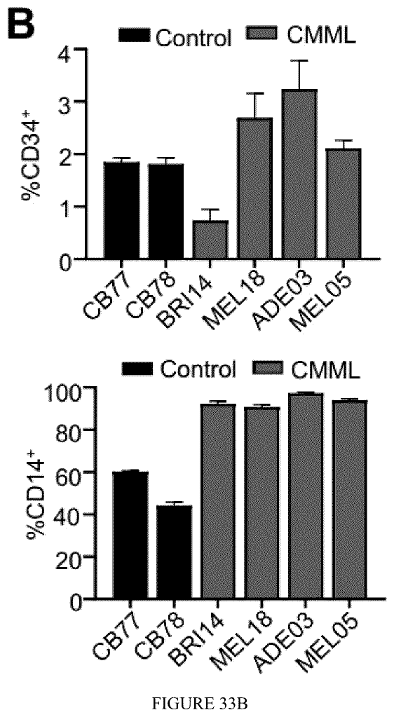

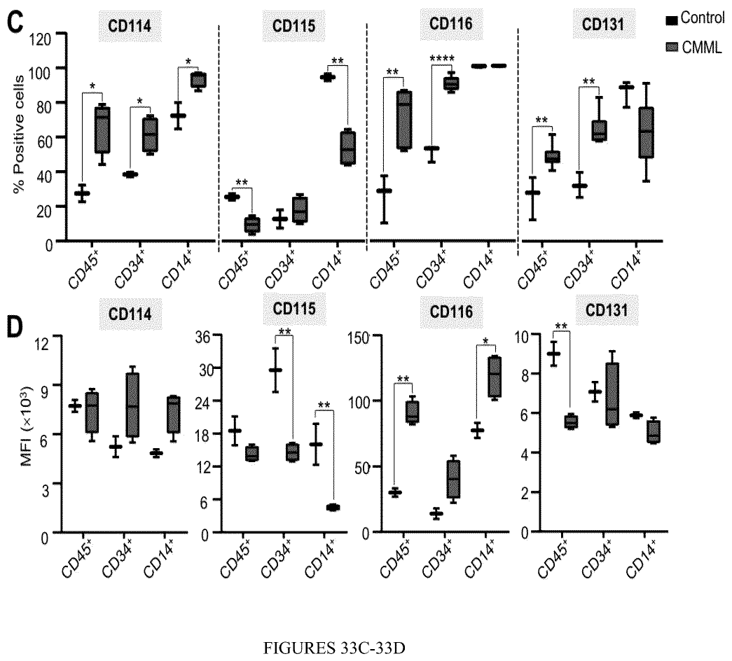

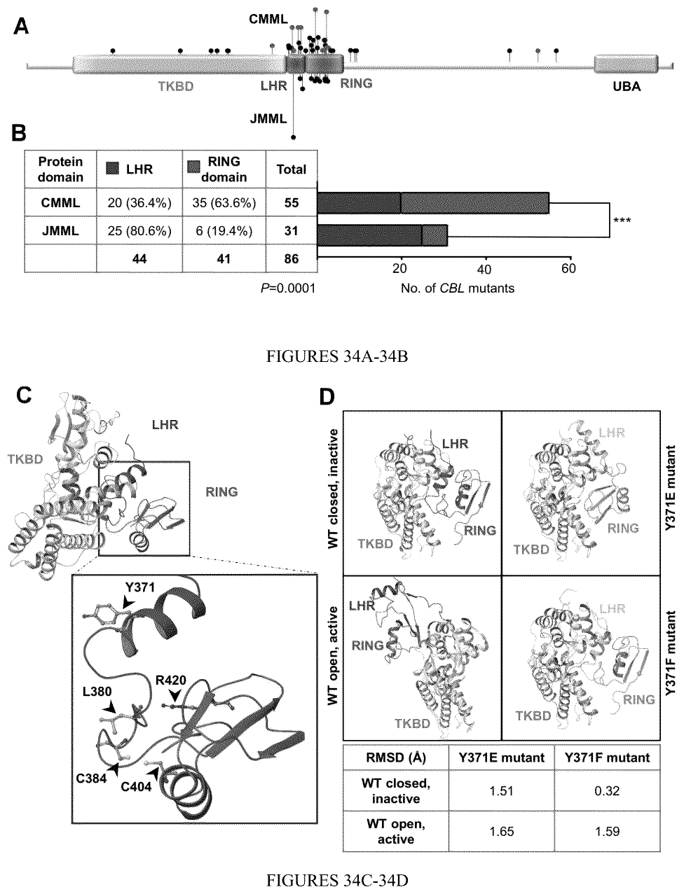

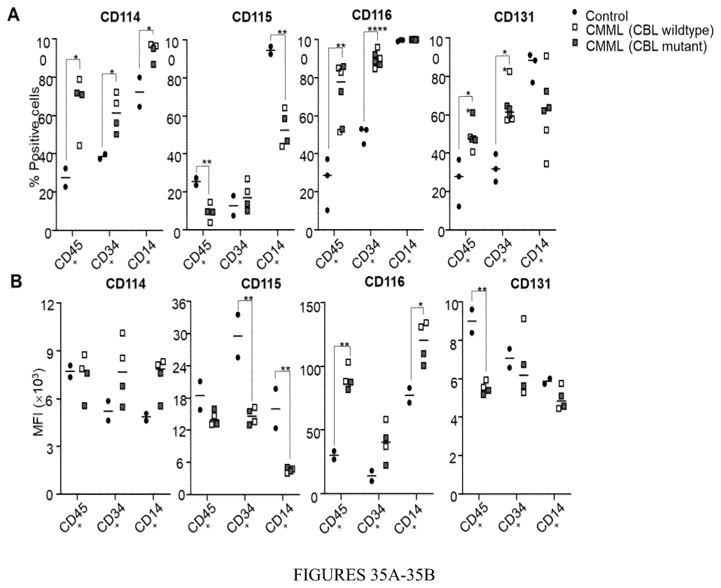

Targeted next-generation sequencing was performed on 24 de novo CMML baseline patient samples from the PREACH-M trial. Overall, RAS pathway (KRAS, NRAS, PTPN11, CBL) mutations were detected in 18/24 (75%) patients with CBL mutations detected in 11/24 (45.9%) patients (Tables 3 and 4). Consistent with previous findings where RAS pathway mutations were linked to the proliferative variant of CMML, patients with RAS pathway mutations had increased BM blast percentage (9.2±1.1 vs. 5.3.±1.4%, P=0.05), white cell count (WCC) (30.4±6.0 vs. 13.8±5.3×10⁹/L, P=0.02), neutrophil absolute count (15.2±3.3 vs. 7.2±3.4×10⁹/L, P=0.03), monocyte absolute count (7.8±1.7 vs. 3.1±1.1×10⁹/L, P=0.03) and spleen length (15.2±0.9 vs. 12.0±0.5 cm, P=0.04) compared to patients wildtype for any RAS pathway mutations (Table 3). Furthermore, patients with CBL mutations were also noted to have increased BM blast percentage (10.1±1.5 vs. 5.3±1.4%, P=0.05), WCC (26.8±5.9 vs. 13.8±5.2×10⁹/L, P=0.05), and spleen length (15.2±1.1 vs. 12.0±0.5 cm, P=0.03) compared to CBL wildtype patients without RAS pathway mutations (Table 4 and FIGS. 31A-31F). 10/11 (90.9%; P=0.03) patients with CBL variants presented with splenomegaly (Table 2B). This underscores the strong proliferative phenotype conferred by mutations in CBL in CMML. Importantly, 8/11 (72.7%) CBL variants were in cases classified as myeloproliferative-CMML (MP-CMML) based on WCC (FIG. 31G), and 9/11 (81.8%) classified as CMML-1 or -2 based on BM blast percentage (FIG. 31H), linking CBL mutations not just to a proliferative phenotype but to more advanced stages of the disease, and therefore, to increased risk of progression to AML.

TABLE 3
Clinical characteristics, complete blood examination and bone marrow analyses of
CMML patients in the PREACH-M trial stratified as RAS pathway (KRAS, NRAS,
PTPN11, CBL) mutant vs. wildtype

| | | RAS pathway | RAS pathway | |
|---|---|---|---|---|
| | Total | wildtype | mutant | |
| Variable | (n = 24) | (n = 6) | (n = 18) | P-value |
| Gender | | | | |
| Male, n (%) | 13 (54%) | 2 (15%) | 11 (85%) | 0.3572 |
| Female, n (%) | 11 (46%) | 4 (36%) | 7 (64%) | |
| Age (years) | | | | |
| Mean (range) | 72 (56-86) | 73 (56-86) | 70 (56-79) | 0.1424 |
| WHO classification | | | | |
| CMML-0, n (%) | 4 (17%) | 1 (17%) | 3 (17%) | 0.8215 |
| CMML-1, n (%) | 13 (54%) | 4 (67%) | 9 (50%) | |
| CMML-2, n (%) | 7 (29%) | 1 (17%) | 6 (33%) | |
| MD-CMML, n (%) | 9 (38%) | 4 (67%) | 5 (28%) | 0.1501 |
| MP-CMML, n (%) | 15 (63%) | 2 (33%) | 13 (72%) | |
| BM Blast (%) | | | | |
| Mean (range) | 7.3 (1.0-17.0) | 5.3 (1.0-10.3) | 9.2 (2.0-17.0) | 0.0493 |
| WCC (×10⁹/L) | | | | |
| Mean (range) | 22.1 (4.9-103.3) | 13.8 (4.9-36.8) | 30.4 (6.9-103.3) | 0.0224 |
| Hb (g/L) | | | | |
| Mean (range) | 107 (79-143) | 104 (79-124) | 109 (82-143) | 0.3426 |
| PLT (×10⁹/L) | | | | |
| Mean (range) | 82 (7-219) | 91 (7-219) | 73 (18-192) | 0.3235 |
| Neutrophils (10⁹/L) | | | | |
| Mean (range) | 11.2 (1.67-47.5) | 7.2 (1.7-23.3) | 15.2 (3.0-47.5) | 0.0329 |
| Monocytes (10⁹/L) | | | | |
| Mean (range) | 5.4 (0.7-32.8) | 3.1 (1.0-8.0) | 7.8 (0.7-32.8) | 0.0267 |
| CRP (mg/L) | | | | |
| Mean (range) | 4.9 (0.6-18.9) | 5.2 (1.7-18.9) | 4.5 (0.6-17) | 0.4796 |
| Spleen craniocaudal length (cm) | | | | |
| Mean (range) | 13.6 (9.8-20.6) | 12.0 (10.8-13.8) | 15.2 (9.8-20.6) | 0.0400 |

n number of patients; BM bone marrow; WCC white blood cell count; Hb hemoglobin; PLT platelet; CRP C-reactive protein; MD-CMML myelodysplastic CMML; MP-CMML myeloproliferative CMML.
Mann-Whitney test was applied to continuous and Fisher's exact test to categorical data for statistical analysis where P < 0.05 was statistically significant.
WHO Classification:
(i) Based on BM blast %: CMML-0 PB <2%, BM <5%; CMML-1 PB 2-4%, BM 5-9%, CMML-2 PB >5%, BM 10-19%.
(ii) Based on WCC: MD-CMML WCC <13 × 10⁹/L, MP-CMML WCC >13 × 10⁹/L

TABLE 4
Clinical characteristics, complete blood examination and bone marrow analyses of
CMML patients in the PREACH-M trial stratified as CBL mutant vs. RAS
pathway wildtype pathway CBL mutant

| | | RAS pathway | | |
|---|---|---|---|---|
| | Total | wildtype | CBL mutant | |
| Variable | (n = 17) | (n = 6) | (n = 11) | P-value |
| Gender | | | | |
| Male, n (%) | 8 (47%) | 2 (33%) | 6 (55%) | 0.6199 |
| Female, n (%) | 11 (65%) | 4 (67%) | 5 (45%) | |
| Age (years) | | | | |
| Mean (range) | 72 (56-86) | 73 (56-86) | 71 (56-79) | 0.2539 |
| WHO classification | | | | |
| CMML-0, n (%) | 3 (18%) | 1 (17%) | 2 (18%) | 0.7964 |
| CMML-1, n (%) | 9 (53%) | 4 (67%) | 5 (45%) | |
| CMML-2, n (%) | 5 (29%) | 1 (17%) | 4 (36%) | |
| MD-CMML, n (%) | 7 (41%) | 4 (67%) | 3 (27%) | 0.1618 |
| MP-CMML, n (%) | 10 (59%) | 2 (33%) | 8 (73%) | |
| BM Blast (%) | | | | |
| Mean (range) | 7.7 (1.0-17.0) | 5.3 (1.0-10.3) | 10.1 (4.0-17.0) | 0.0457 |
| WCC (×10⁹/L) | | | | |
| Mean (range) | 20.3 (4.9-74.1) | 13.8 (4.9-36.8) | 26.8 (6.9-74.1) | 0.0462 |
| Hb (g/L) | | | | |
| Mean (range) | 106 (79-128) | 104 (79-124) | 107 (91-128) | 0.4893 |
| PLT (×10⁹/L) | | | | |
| Mean (range) | 89 (7-219) | 91 (7-219) | 88 (27-192) | 0.5000 |
| Neutrophils (10⁹/L) | | | | |
| Mean (range) | 10.6 (1.7-45.2) | 7.2 (1.7-23.3) | 14.0 (3.0-45.2) | 0.0608 |
| Monocytes (10⁹/L) | | | | |
| Mean (range) | 4.3 (0.7-12.2) | 3.1 (1.0-8.0) | 5.6 (0.7-12.2) | 0.0859 |
| CRP (mg/L) | | | | |
| Mean (range) | 4.4 (1.7-18.9) | 5.2 (1.7-18.9) | 3.6 (0.7-7.3) | 0.4708 |
| Spleen craniocaudal length (cm) | | | | |
| Mean (range) | 13.6 (10.2-20.6) | 12.0 (10.8-13.8) | 15.2 (10.2-20.6) | 0.0308 |

n number of patients; BM bone marrow; WCC white blood cell count; Hb hemoglobin; PLT platelet; CRP C-reactive protein; MD-CMML myelodysplastic CMML; MP-CMML myeloproliferative CMML
Mann-Whitney test was applied to continuous and Fisher's exact test to categorical data for statistical analysis where P < 0.05 was statistically significant.
WHO Classification:
(i) Based on BM blast %: CMML-0 PB <2%, BM <5%; CMML-1 PB 2-4%, BM 5-9%, CMML-2 PB >5%, BM 10-19%.
(ii) Based on WCC: MD-CMML WCC <13 × 10⁹/L, MP-CMML WCC >13 × 10⁹/L

FIGS. 31A-31H show CBL mutants are associated with proliferative features, increased BM blast percentage, leukocytosis and splenomegaly. FIGS. 31A-31F show clinical characteristics of PREACH-M cohort at screening, stratified based on the detection of CBL mutation. FIG. 31G shows MD-, MP-CMML classification based on WCC^{α} FIG. 31H shows CMML-0, -1, -2 classifications based on BM blast percentage^{β}

### 2016 WHO Classification:

^{α}Based on WCC: MD-CMML WCC<13×10⁹/L, MP-CMML WCC>13×10⁹/L
^{β}Based on BM blast %: CMML-0 PB<2%, BM<5%; CMML-1 PB 2-4%, BM 5-9%, CMML-2 PB>5%, BM 10-19%.
Bars represent mean±standard error of mean. Mann-Whitney test used to determine statistical significance, where P<0.05 was statistically significant. *P<0.05.
[BM bone marrow; WCC white cell count; CRP C-reactive protein; MD-CMML myelodysplastic-CMML; MP-CMML myeloproliferative-CMML, wt wildtype, mut mutant]
CBL Mutations Co-Occur Frequently with TET2

Mutation screening of the targeted panel revealed that CBL mutation most frequently co-occur with TET2 mutation (82%), followed by ASXL1 (64%) and SRSF2 (55%) (FIGS. 32A and 32B). Additionally, a significant positive Pearson's correlation coefficient was noted between overall TET2 and CBL variant allele frequency (VAF) percentages (r²=0.68, P=0.002) (FIG. 32D), suggesting they are present in the same clone. Conversely, there was an inverse correlation between the VAF percentages of CBL vs., RAS pathway genes (r²=0.29, P=0.35) (FIG. 32E), indicating they are unlikely to co-occur in the same cell.

FIGS. 32A-32E show CBL mutants frequently co-occur with TET2 mutants with strong correlation between CBL and TET2 VAF percentages and are associated with a complex subclonal architecture. FIG. 32A shows an oncoplot for the PREACH-M cohort (n=24). Mutation groups are shown in rows with each individual patient represented by a column. The presence of a mutation is indicated by the red or blue colored bars. Age category of the patients indicated by the black and grey bars and sex of patients by the green and gold bars. FIG. 32B shows co-occurrence of CBL mutant CMML (n=11) with other mutations by percentage, compared to CBL wildtype (n=13). FIG. 32D shows the number of cases where more than one variant of CBL, NRAS, KRAS or PTPN11 mutation was detected. FIG. 32C shows Pearson's correlation coefficient between TET2 and CBL VAF percentages, where P<0.05 was statistically significant. Pearson's correlation coefficient between the VAF percentages of CBL vs. RAS pathway genes. (data not shown) FIG. 32E shows VAF percentages of CBL variants detected in each patient with CBL mutation. [VAF variant allele frequency; V variant]

### Multiple CBL Mutant Subclones Found in CMML

In 7/11 (63.6%) patients with CBL mutation, more than one CBL variant can be detected (FIGS. 32C and 32E). Of these, 2 patients had 3 variants while 5 patients had 2 variants (FIG. 32E). In comparison, 3/7 and 2/5 cases harbored more than one KRAS and NRAS variants, respectively (FIG. 32D), with no more than 2 variants detected.

### CMML Linked to High CD116 and CD131 in the Progenitor Subpopulation

As CMML is a disease characterized by upregulation of inflammatory cytokines and expansion of pro-inflammatory granulocyte-macrophage-like progenitor cells and monocytes with enhanced cytokine receptor signaling, the immunophenotype of primary patient samples was analyzed focusing on cytokine receptor expression (granulocyte colony-stimulating factor receptor, CD114; macrophage colony-stimulating factor receptor, CD115; and the heterodimeric granulocyte-macrophage colony-stimulating factor receptor comprising the alpha subunit (GMRα), CD116 and the beta common subunit (βc), CD131) in the CD45⁺ MNCs, CD34⁺ hematopoietic stem and progenitor cells (HSPCs) and CD14⁺ monocytes (gating strategy outlined in FIG. 33A). CMML patient samples had higher percentage of CD14⁺ cells compared to normal donors, consistent with expansion of monocytes and clinical presentation of the disease (FIG. 33B).

FIGS. 33A-33B show CMML have an increased percentage of CD116 and CD131 positive CD34⁺ stem and progenitor cells. FIG. 33A shows flow cytometry analysis of a representative CMML sample and healthy control stained with anti-CD45, -CD34, -CD14 and -CD16, and gating strategy used to define CD45⁺ mononuclear cells, CD34⁺ stem and progenitor cells, and CD14⁺ monocytes. FIG. 33B shows percentage of CD34⁺ stem and progenitor cells and CD14⁺ monocytes in CMML samples (n=4) vs. healthy control (n=2). The expression of CD114, CD115, CD116 and CD131 in CMML samples (n=4-6) vs. control (n=2-3) in CD45⁺, CD34⁺ and CD14⁺ subpopulations, expressed as percentage of positively stained cells (FIG. 33C) and MFI (FIG. 33D) compared to control (cord blood or peripheral blood mononuclear cells from healthy donors). Bars represent mean±standard deviation in FIG. 33B. Box and whiskers graphs were plotted with min and max in FIG. 33C and FIG. 33D. Unpaired Student's t-test between CMML vs. healthy control used to determine statistical significance, where P<0.05 was statistically significant. *P<0.05, **P<0.01, ***P<0.001, ****P<0.0001. [MFI mean fluorescence intensity]

Our data revealed that the percentage of cells in CMML patient samples expressing CD116 (GMRα) was significantly higher in the total MNC population compared to control, and this was most pronounced in the CD34⁺ HSPC subpopulation (89.7±1.6 vs. 50.3±2.7%; P=0.000003) (FIG. 33C). In contrast, the percentage of CD116 expressing cells was similar between healthy and CMML-derived CD14⁺ monocytes (FIG. 33C). Interestingly, the density of CD116 expression represented by mean fluorescence intensity (MFI) was upregulated in our CMML cohort vs. healthy controls (CD45⁺ 90.3±4.6×10³ vs. 30.0±3.2×10³, P=0.001; CD34⁺ 40.2±7.4×10³ vs. 13.9±4.1×10³, P=0.08; CD14⁺ 118.9×10³±8.1×10³ vs. 77.4×10³±5.8×10^{3;} P=0.03) (FIG. 33D). An increase in the percentage of CD131 expression in the MNCs also was noted, particularly in the CD34⁺ HSPCs in CMML compared to control (64.3±3.8 vs. 32.1±4.1%; P=0.001), although not in terms of MFI (FIGS. 33C and 33D). A difference in receptor expressions between CBL mutant and CBL wildtype CMML patient samples was not observed (FIGS. 35A-35B, which show cytokine receptor CD114, CD115, CD116 and CD131 expression by percentage positive cells (FIG. 35A) and mean fluorescence intensity (MFI) (FIG. 35B)). In FIGS. 35A-35B: The expression of CD114, CD115, CD116 and CD131 in CMML samples (n=4-6) vs control (n=2-3) in CD45+, CD34+ and CD14+ subpopulations. CBL mutant CMML represented by red squares, wildtype by clear squares. Bars represent medians. Unpaired t-test was applied for statistical analysis, where P<0.05 was considered significant. *P<0.05, **P<0.01, ***P<0.001, ****P<0.0001.

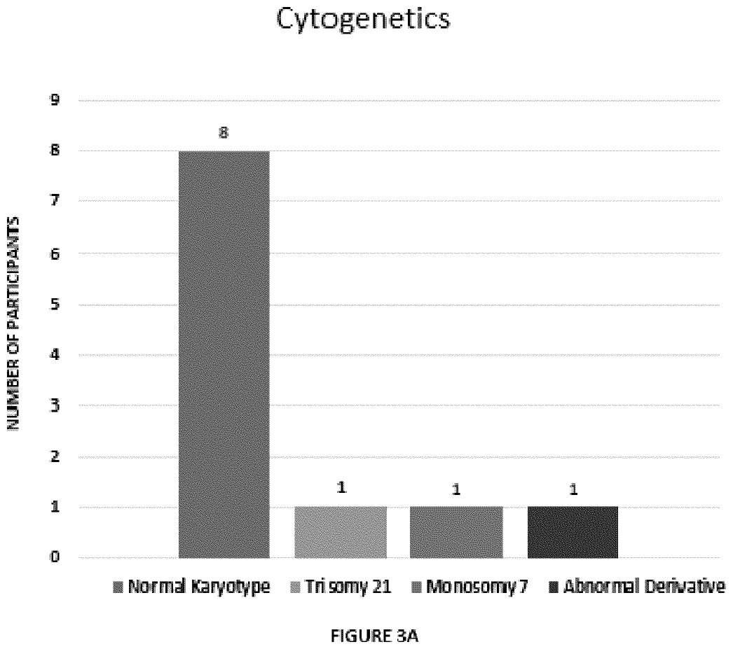

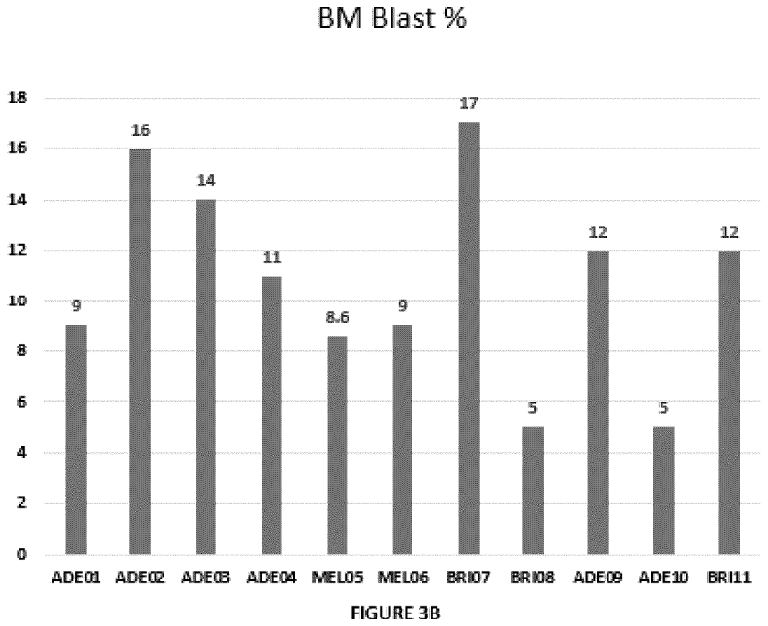

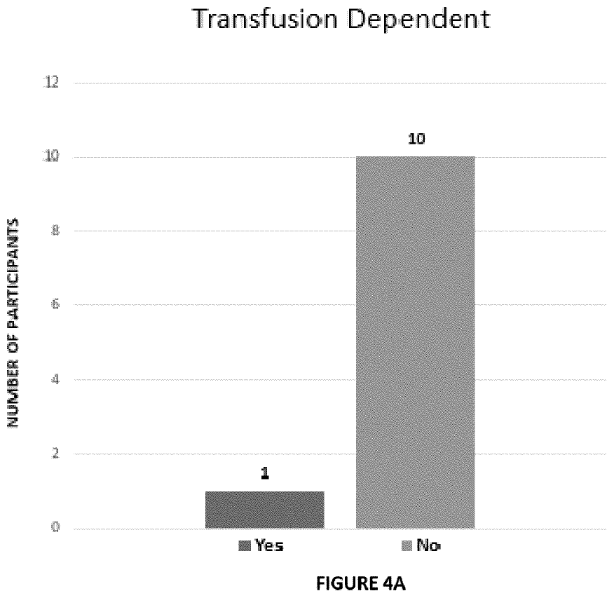

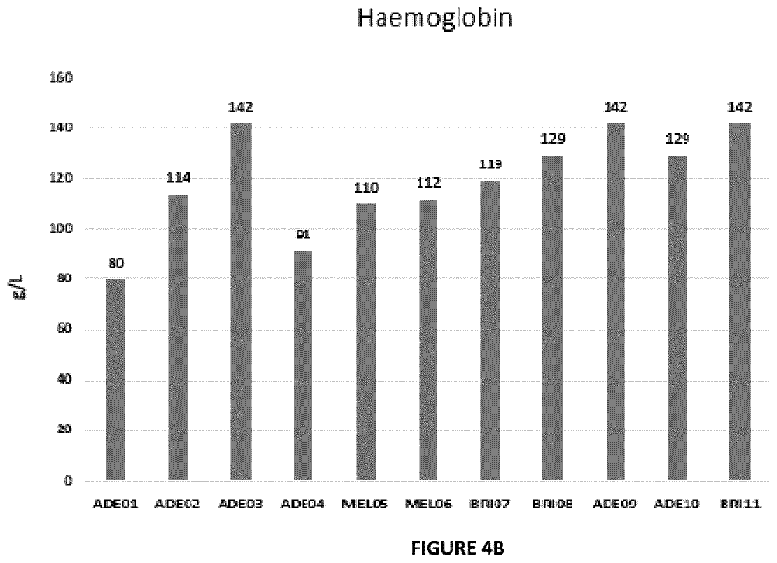

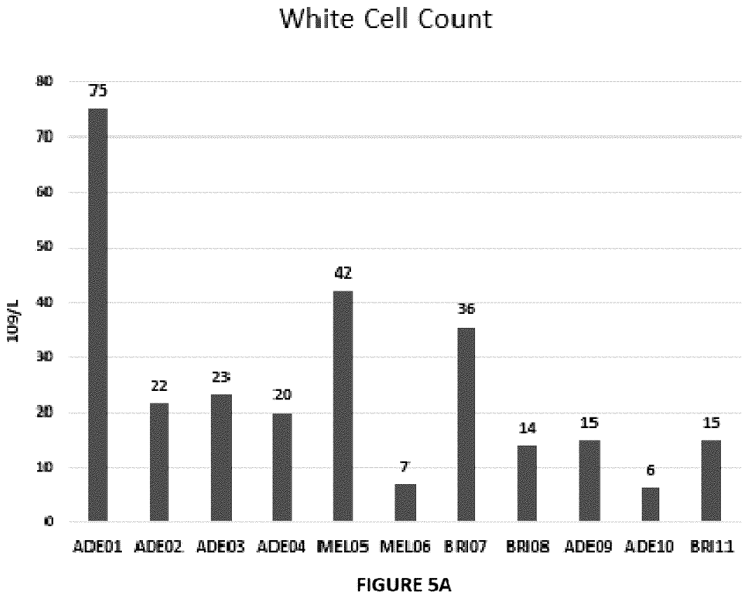

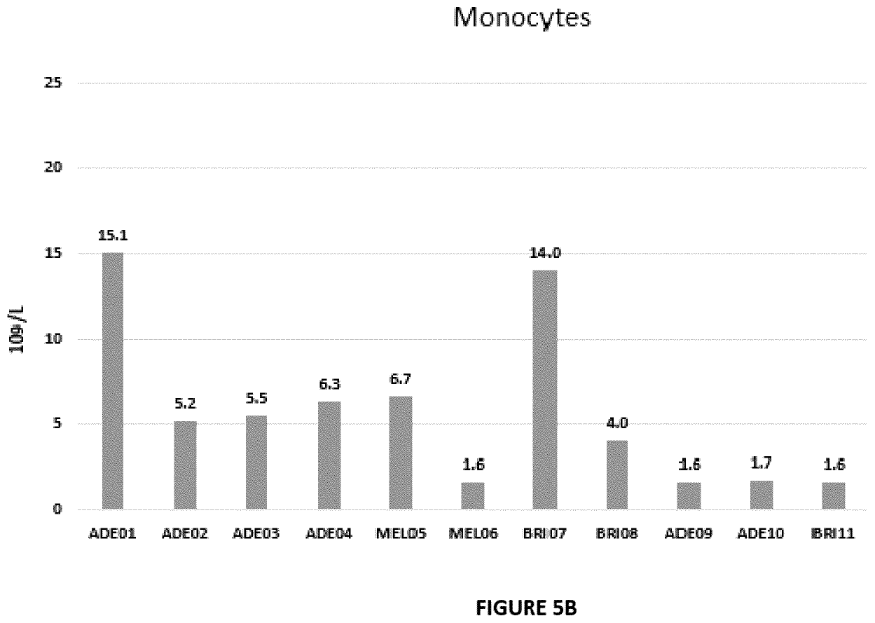

Increases in the percentage of CD114⁺ cells were also noted across all CMML cell populations (CD45⁺ P=0.03; CD34⁺ P=0.04; CD14⁺ P=0.02), with no difference in the MFI (FIGS. 3C and D). In contrast, the percentage of CD115⁺ cells was notably lower in the CMML CD45⁺ MNCs (P=0.01) and CD14⁺ monocytes (P=0.005), with reductions in MFI seen in both CD34⁺ (P=0.005) and CD14⁺ (P=0.007) populations (FIGS. 33C and 33D)

### CBL Mutations are Enriched in the RING Domain in CMML Compared to JMML

The new mutation data from our cohort was then combined with the publicly available data in COSMIC to assess which domains of Cbl were commonly perturbed. It was found that CBL mutations in CMML and JMML are concentrated within the coding sequence of the LHR and RING domain of Cbl (FIG. 34A). Furthermore, it was noted that in CMML, mutations most frequently occur within the RING domain (amino acid residues 381-435) contrary to JMML, where mutations within the LHR (amino acid residues 353-380) are most common (P<0.0001) (FIG. 34B).

FIGS. 34A-34 show CBL mutations hotspots in CMML cluster in the RING domain, unlike in JMML where they more commonly occur within the LHR. FIG. 34A shows a lollipop representation of mutation hotspots in key Cbl functional domains; contrasting frequencies detected in CMML (top) and JMML (bottom). Mutations detected in patients in the PREACH-M study are highlighted in red. FIG. 34B shows contingency table of CBL mutation hotspots within the LHR and RING domain of the protein. Data sourced from COSMIC and the PREACH-M study. FIG. 34C shows tertiary protein structure of native, inactive Cbl wildtype (PDB 2Y1M). The TKBD is colored beige, LHR blue and RING domain red. Amino acid residues of the top 6 mutation hotspots are indicated in green; Tyrosine371 (Y371), Leucine380 (L380), Cysteine384 (C384), Cysteine396 (C396), Cysteine404 (C404) and Arginine420 (R420). FIG. 34D shows X-ray structures of wildtype Cbl in unphosphorylated, inactive state and in closed conformation (PDB 2Y1M) (top left); wildtype Cbl in Y371 phosphorylated, active state and in open conformation (PDB 4A4C) (bottom left); mutant Cbl Y371E (PDB 5HKX) (top right) and mutant Cbl Y371F (PDB 5J3X) (bottom right). The TKBD is colored beige, LHR of wildtype blue, LHR of mutant cyan, RING domain of wildtype red, RING domain of mutant pink. Statistical analysis was performed using two-sided Fisher's exact test, where P<0.05 was statistically significant. ****P<0.0001 [TKBD=tyrosine kinase binding domain; LHR=linker helix region; RING=RING domain; UBA=ubiquitin-association domain; WT=wildtype; MUT=mutant; RMSD=root mean square deviation (distanced-based measure of protein structure similarity)]

The 6 most common CBL mutations in all hematopoietic and lymphoid malignancies occur in codons affecting amino acid residues 371, 380, 384, 396, 404, 420 (Table 5 and FIG. 34C). In the PREACH-M cohort in particular, mutations in residue 384 were detected 3 patients, 371 in 2 patients, 380 in 2 patients, 404 and 420 in 1 patient, respectively. In CMML, missense substitutions cysteine 404 to tyrosine (C404Y) (11%), and arginine 420 to glutamine (R420Q) (10%) were most common, while in JMML, tyrosine 371 to histidine (Y371H) was most common (46%) (Table 5).

TABLE 5
Six most commonly occurring CBL mutations in hematopoietic and
lymphoid malignancies, CMML and JMML (source: COSMIC; last
accessed 15 Feb. 2024) compared to the PREACH-M cohort in our study.

| | Linker helix RING domain | | | | | | |
|---|---|---|---|---|---|---|---|
| | region (LHR) | | | | | | Variants |
| Protein structural | Amino acid residue | | | | | | detected, |
| domain | 371^{a} | 380^{b} | 384^{c} | 396^{d} | 404^{e} | 420^{f} | n |
| All hematopoietic | 49 | 33 | 24 | 23 | 36 | 45 | 526 |
| and lymphoid | (9%) | (6%) | (5%) | (4%) | (7%) | (9%) | |
| malignancies, n (%) | | | | | | | |
| CMML, n (%) | 8 | 8 | 7 | 7 | 16 | 15 | 120 |
| | (7%) | (7%) | (6%) | (6%) | (13%) | (13%) | |
| JMML, n (%) | 24 | 1 | 5 | 1 | 1 | 0 | 46 |
| | (52%) | (2%) | (11%) | (2%) | (2%) | | |
| PREACH-M cohort, | 2 | 2 | 3 | 0 | 1 | 1 | 17 |
| n (%) | (12%) | (12%) | (18%) | | (6%) | (6%) | |

Most common missense substitutions:
^{a}Y371H (n = 21) and ^{c}C384R (n = 5) in JMML;
^{b}L380P(n = 8), ^{d}C396R (n = 3), ^{e}C404Y (n = 13) and ^{f}R420Q (n = 12) in CMML.

### Mutations at Residue 371 Within the LHR Can Result in Novel Conformational Change

Finally, comparative structural alignments of a number of Cbl structures resolved by X-ray diffraction publicly available via the Protein Data Bank (PDB) were performed. Wildtype Cbl protein in the inactive conformation (LHR unphosphorylated) (PDB 2Y1M) was compared against the active conformation (LHR phosphorylated) (PDB 4A4C) (FIG. 34D), had a root mean square deviation (RMSD) of 1.52 Å, indicating that a conformational change takes place when Cbl becomes activated by phosphorylation. Further, PDB structures 2Y1M and 4A4C were also compared with structures comprising tyrosine 371 to glutamic acid (Y371E) (PDB 5HKX) and tyrosine 371 to phenylalanine (Y371F) (PDB 5J3X) LHR mutations. Interestingly, it was inferred that the Y371E mutant Cbl possesses an entirely different conformation to either the inactive or active wildtype Cbl (RMSD 1.51 Å and 1.65 Å, respectively). It was noted that replacement of the polar, bulky tyrosine with the negatively charged glutamic acid (Y371E) resulted in perturbation of the LHR-TKBD interface and subsequent total displacement of the LHR and RING domain compared to both inactive and active wildtype Cbl (FIG. 34D). In contrast, when tyrosine was replaced with a structurally similar residue phenylalanine (Y371F), the LHR-TKBD interface was unperturbed, and the mutant closely mimicked the native, inactive state of wildtype Cbl (RMSD 0.32 Å) and not the active state (RMSD 1.59 Å) (FIG. 34D). The potential implications of a conformational change in Cbl mutant could include the impairment of Cbl activity and subsequent detrimental effects following prolonged RTK signaling. More importantly, discovering mutant conformations that are different to wildtype provides a therapeutic avenue via selective targeting of Cbl mutant proteins for degradation.

### Discussion

Our data, gleaned from patients enrolled in a prospective multicenter interventional study, highlight several clinical and molecular features of CBL mutants in CMML. Our results are generally consistent with previous studies that performed next generation sequencing in CMML patients but a higher frequency of CBL variants (11 of 24 patients, 46%) was noted than others report (12.8%), possibly due to strict trial eligibility criteria (higher white cell count or cytopenia). CBL variants were associated with a myeloproliferative phenotype, including higher white cell count and splenomegaly with many patients having increased blasts at diagnosis, similar to patients with other RAS pathway mutations. Notably, many patients harbored multiple CBL subclones (intrapatient molecular heterogeneity) that was not observed to the same extent for other RAS pathway mutations. This may be significant because subclonal abundance, especially a branched pattern of clonal evolution, is associated with a favorable outcome in acute myeloid leukemia.

A strong overlap and clonal correlation was observed between CBL mutations and TET2 mutation. Another study has found a modest association of TET2 with CBL mutations (r<0.25; P<0.1), despite the high frequency of TET2 mutation overall. A number of in vivo murine studies have highlighted a role for TET2 in suppressing innate immune signaling in monocytes, with TET2 mutant monocytes showing enhanced pro-inflammatory responses to stimuli such as lipopolysaccharide. It is plausible that the CBL mutation serves to further amplify innate immune signaling by preventing ubiquitination and turnover of cytokine and Toll-like receptors but the exact cellular compartments within which this occurs is not defined. Thus, it is significant that a high percentage of CBL mutant CMML CD34⁺ HSPCs express GMRα (CD116, 89.7%) and its partner subunit, βc (CD131, 64.3%), suggesting that a substantial proportion of CD34⁺ cells are primed to respond to the cytokine granulocyte-macrophage colony-stimulating factor (GM-CSF). Indeed, it is known from previous studies that CMML display hypersensitivity to GM-CSF akin to JMML, especially in cases that have RAS pathway mutations. In contrast, the percentage expression of the receptor for macrophage colony stimulating factor (CD115) was decreased in CMML progenitors compared to controls, indicating this alternate monocyte cytokine is unlikely to be driving the disease. An increased percentage of CD114 CMML also was noted but not MFI compared to controls, although not to the same extent as CD116, indicating CMML progenitors may also respond to granulocyte-colony stimulating factor.

Previous studies with GM-CSF neutralizing antibody and prior work with the GM-CSF E21R antagonist and successful engraftment of CMML patient samples in mice transgenic for human GM-CSF provide strong evidence that GM-CSF is an essential growth factor for CMML in vitro and in vivo. Our findings that CD116 is upregulated in the CD34⁺ HSPCs is interesting as it raises the question of the effect of GM-CSF on the leukemia-initiating cell population. This is in agreement with a recent study using single cell RNAseq to map the differentiation trajectories of CD34⁺ HSPCs in CMML primary patient samples where the authors also showed the upregulation of CD116 in a cell cluster enriched for granulocyte/monocyte progenitor-like inflammatory HSPCs with self-renewal capacity in CMML patients with a monocyte-biased differentiation trajectory. Recently, it was found that interleukin-3 (IL-3) receptor stoichiometry is a critical determinant in cell fate and IL-3 receptor overexpression in leukemia stem cells leads to biased activation of distinct transcriptional programs and signaling pathways to drive stemness programs vs. cell differentiation. This propels us to hypothesize that although GM-CSF has been mostly associated with the proliferation and differentiation of hematopoietic progenitors and mature cells, it is possible that aside from the cytokine hypersensitivity previously shown, GM-CSF may also have unique signaling and effects on stem cell maintenance and function in CD116-overexpressing CD34⁺ HSPCs for disease initiation and generation of the pro-inflammatory phenotype associated with this disease. Future studies should examine the kinetics of receptor turnover and phosphorylation peak and attenuation in this primary population, the signaling pathways involved and determine whether CD116 can be used to distinguish between CBL mutant leukemia stem cells and healthy HSPCs. Indeed, the effects of anti-CD116 or anti-GM-CSF therapies (or in combination), on these populations warrant further investigation.

Cbl adopts a closed and open conformation dependent on Y371 phosphorylation to allow for the binding of ubiquitin conjugating enzyme E2. Thus, it follows that the loss of this key tyrosine residue in position 371 within the LHR could have dramatic implications for the conformation and activity of Cbl. Tyrosine 371 is in a buried environment and makes multiple van der Waals interactions and hydrogen bonds with other residues in the hydrophobic pocket of the TKBD, playing a structural role in maintaining the integrity of the LHR-TKBD interface, and importantly, in keeping Cbl in a closed conformation, autoinhibited state. When Y371 is phosphorylated, an open conformation is adopted, and autoinhibition is abolished leading to Cbl becoming a more active ligase. Our analysis of the X-ray structures demonstrates that Cbl conformation is sensitive to the amino acid residue at position 371. Indeed, our inference following comparative structural alignments is that, depending on the nature of the substituting residue, mutation at position 371 (such as Y371E, PDB 5HKX) not only results in the impairment of phosphorylation-dependent activation, but can also yield an entirely novel conformation of Cbl, different from both the inactive (unphosphorylated, closed) and active (phosphorylated, open) conformations. With this conformational drift, the RING domain and E2 enzyme cannot be in sufficient proximity to the substrate binding site of the TKBD for effective ubiquitination. Thus, ubiquitination and degradation of activated RTKs would be predicted to occur less efficiently, resulting in sustained downstream signaling that may contribute to oncogenicity and disease progression. In contrast, mutations that do not perturb the LHR-TKBD interaction (such as Y371F, PDB 5J3X) would mimic the conformation of native, wildtype Cbl, albeit no longer capable of increased catalytic efficiency due to loss of the phosphorylation site. This is consistent with early evidence that Cbl Y371 mutants can exist in different states of activity depending on the chemical nature of the amino acid substitution.

In CMML, CBL mutations were found in both the LHR and RING domains of the protein but with significant enrichment for mutations in the RING domain compared to JMML. The RING domain determines the specificity of Cbl E3 for its cognate E2 enzyme, recognizes lysines to be ubiquitinated and serves as a scaffold for optimal orientation for ubiquitin transfer between E2 and its substrate RTK but the structure of Cbl RING domain mutations has not been determined. Elucidation of distinct mutant Cbl conformations is significant because new drug development strategies could employ proteolysis targeting chimera (PROTAC) technology for targeted protein degradation of mutant Cbl with conformations different from wildtype. Conversely, a new Cbl-b inhibitor C7683 currently in phase I clinical trials for advanced solid tumor malignancies, designed to keep wildtype Cbl-b locked in an inactive state, may partially mimic LHR Cbl mutations and thus should be used with caution in patients with clonal hematopoiesis, CMML or JMML.

A limitation of this study is the relatively small number of CBL mutant positive cases, reflecting the rarity of CMML. Nevertheless, certain clinical and molecular features are consistent across the cohort and are congruent with data available in COSMIC, implying that CBL mutant CMML may have a characteristic phenotype. Studies with larger cohorts are required to distinguish CBL mutation CMML from other RAS pathway mutations such as NRAS, KRAS and PTPN11. To date, one study using serial VAF measurements did not show significant change in clone size for CMML patients, including CBL clones, treated with azacitidine alone suggesting epigenetic effects, rather than mutation-specific effects, were linked to therapeutic benefit. Future work should examine the effect of CD116-targeted immunotherapy on the clonal dynamics of CBL RING domain vs. LHR mutants.

Having described certain embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

1. A method for treating a subject having chronic myelomonocytic leukemia (CMML), the method comprising:
a) identifying a RAS pathway mutation in tumor cells of the subject, wherein the RAS pathway mutation is a NRAS, KRAS, PTPN-11 and/or CBL mutation;
b) identifying a dominant CBL mutation of CBL variant allele frequency of from <5% to >10%; and
c) administering to the subject identified in steps (a) and (b) or solely in step (b) a therapeutically effective amount of an anti-hGM-CSF antibody.

2. The method of claim 1, further comprising administering a therapeutically effective amount of a second therapeutic agent, wherein the second therapeutic agent is a hypomethylating agent or a chemotherapy drug.

3. The method of claim 2, wherein the hypomethylating agent is administered for five to seven days starting on day one of administration of the anti-hGM-CSF antibody.

4. The method of claim 2, wherein the chemotherapy drug is hydroxyurea and wherein the hypomethylating agent is selected from the group consisting of azacytidine, decitabine, and a combination of decitabine and cedazuridine.

5. The method of claim 1, further comprising identifying an increased percentage of CD116 and CD131 in CD34⁺ stem and progenitor cells in the subject compared to a healthy subject or further comprising identifying an increased percentage of CD14⁺ cells in the subject compared to a healthy subject.

6-7. (canceled)

8. The method of claim 1, wherein the anti-hGM-CSF antibody is selected from the group consisting of lenzilumab, Namilumab, Otilimab, Gimsilumab, and TJM2 (TJ003234).

9. (canceled)

10. The method of claim 2, wherein the anti-hGM-CSF antibody is selected from the group consisting of lenzilumab, Namilumab, Otilimab, Gimsilumab, and TJM2 (TJ003234).

11. (canceled)

12. The method of claim 8, wherein the anti-hGM-CSF antibody is administered on day one and day 15 of cycle 1 and on day one only for all subsequent cycles, and wherein the all subsequent cycles consist of a total of 24 cycles, and each cycle consists of 28 days.

13. The method of claim 10, wherein the anti-hGM-CSF antibody is administered on day one and day 15 of cycle 1 and on day one only for all subsequent cycles, and wherein the all subsequent cycles consist of a total of 24 cycles, and each cycle consists of 28 days.

14. The method of claim 1, wherein the subject has a RAS pathway mutation and a TET2 mutation identified in the tumor cells or wherein the subject has a RAS pathway mutation and two TET2 mutation variants identified in the tumor cells.

15. (canceled)

16. The method of claim 8, wherein the anti-hGM-CSF antibody is administered intravenously (IV) at a dose of from 552 mg to 1656 mg, wherein the dose of 552 mg is administered over a 1 hour IV infusion and the dose of 1656 mg is administered over 2 hour(s) IV infusion or is administered at a dose of 552 mg over a 1 hour IV infusion.

17. The method of claim 8, wherein the anti-hGM-CSF antibody lenzilumab is administered intravenously (IV) at a dose of from 552 mg to 1656 mg, wherein the dose of 552 mg is administered over a 1 hour IV infusion and the dose of 1656 mg is administered over 2 hour(s) IV infusion or is administered at a dose of 552 mg over a 1 hour IV infusion.

18. The method of claim 7, wherein the hypomethylating agent is administered subcutaneously at a dose of 75 mg/m².

19. The method of claim 2, wherein the administration demonstrates a complete response (CR) plus partial response (PR) of 50-90% compared to complete response (CR) plus partial response (PR) of 7-21% achieved with sole administration of a hypomethylating agent; and wherein the response is a complete response or a partial response during 6 first cycles.

20. (canceled)

21. The method of claim 19, wherein the CR or PR result in improved survival and progression-free survival at two years after treatment compared to survival and progression-free survival at two years after administration of a hypomethylating agent alone.

22. The method of claim 2, wherein the administration demonstrates/achieves clinical benefit at any point during 24 cycles, wherein the clinical benefit comprises impact on physical and functional capacity of the subject, social well-being of the subject, hematological and non-hematologic safety and combinations thereof.

23. (canceled)

24. The method of claim 22, wherein the hematological safety comprises (a) a decrease in C-reactive protein (CRP) by at least 50% within six months of administering the therapeutically effective amount of the therapeutically effective amount of the anti-hGM-CSF antibody and the hypomethylating agent compared to baseline CRP prior to treatment, and (b) an improvement in hematological parameters; (c) an improved bone marrow response of less than 5% blasts within 12 months or (d) a complete response in subjects having a medium increase in GM-CSF and pro-inflammatory cytokines found in an innate immune response and M1 macrophage activation compared to levels of GM-CSF and pro-inflammatory cytokines and M1 macrophage activation in healthy subjects within 12 months; and/or (e) a decrease in a variant allele frequency (VAF) of at least one identified RAS-pathway mutation, wherein the RAS-pathway mutation is a KRAS and/or CBL mutation; and wherein the clinical benefit comprising impact on the physical capacity of the subject comprises a reduction in splenomegaly.

25-27. (canceled)

28. The method of claim 2, further comprising treating the subject with an allogeneic transplant.

29. A method for treating a subject having chronic myelomonocytic leukemia (CMML), the method comprising:
a) identifying a RAS pathway mutation in tumor cells of the subject, wherein the RAS pathway mutation is a NRAS, KRAS, PTPN-11 and/or CBL mutation;
b) identifying a dominant CBL mutation of CBL variant allele frequency of from <5% to >10%; and
c) administering to the subject identified in steps (a) and (b) or solely in step (b) a therapeutically effective amount of an anti-hGM-CSF antibody and a therapeutically effective amount of a second therapeutic agent.

30. The method of claim 29, wherein the second therapeutic agent is a hypomethylating agent or a chemotherapy drug, wherein the hypomethylating agent is selected from the group consisting of azacytidine, decitabine, and a combination of decitabine and cedazuridine and wherein the chemotherapy drug is hydroxyurea; and wherein the anti-hGM-CSF antibody is selected from the group consisting of lenzilumab, Namilumab, Otilimab, Gimsilumab, and TJM2 (TJ003234).

31. The method of claim 30, wherein the hypomethylating agent is administered for five to seven days starting on day one of administration of the anti-hGM-CSF antibody.

32-57. (canceled)
